(19) United States
(12) Patent Application Publication
(10) Pub. No.: US 2002/0151572 A1
(43) Pub. Date: Oct. 17, 2002

(54) PHARMACEUTICAL USES FOR NOS INHIBITORS

(76) Inventor: JOHN A. LOWE III, STONINGTON, CT (US)

Correspondence Address: PFIZER INC, 150 EAST 42ND STREET, 5TH FLOOR - STOP 49, NEW YORK, NY, 10017-5612, US

(21) Appl. No.: 09/372,352

(22) Filed: Aug. 11, 1999

Related U.S. Application Data
(60) Provisional application No. 60/096,152, filed on Aug. 11, 1998.

(51) Int. Cl. A61K 31/41; A01N 43/82

(52) U.S. Cl. 514/360

(57) ABSTRACT

The present invention relates to new pharmaceutical uses for compounds that exhibit activity as nitric oxide synthase (NOS) inhibitors. Specifically, it relates to the use of NOS inhibitors, particularly selective neuronal NOS (N-NOS) inhibitors: (a) alone or in combination with another active agent for the treatment of psoriasis; (b) in combination with an antiinflammatory agent for the treatment of inflammatory disorders; (c) in combination with a narcotic analgesic (e.g., opiates such as morphine or demerol) for the treatment of pain; (d) alone or in combination with other active agents for the enhancement of cognition; and (e) alone or in combination with other active agents for the treatment of sleep disorders such as apnea, narcolepsy and insomnia.

The present invention relates to new pharmaceutical uses for compounds that exhibit activity as nitric oxide synthase (NOS) inhibitors. Specifically, it relates to the use of NOS inhibitors, particularly selective neuronal NOS (N-NOS) inhibitors: (a) alone or in combination with another active agent for the treatment of psoriasis; (b) in combination with an antiinflammatory agent for the treatment of inflammatory disorders; (c) in combination with a narcotic analgesic (e.g., opiates such as morphine or demerol) for the treatment of pain; (d) in combination with a serotonin-1D (5HT_{1D}) agonist (e.g., eletriptan or sumatriptan) for the treatment of migraine, cluster or other vascular headaches; (e) alone or in combination with other active agents for the enhancement of cognition; and (f) alone or in combination with other active agents for the treatment of sleep disorders such as apnea, narcolepsy and insomnia.

There are three known isoforms of NOS—an inducible form (I-NOS) and two constitutive forms referred to as, respectively, neuronal NOS (N-NOS) and endothelial NOS (E-NOS). Each of these enzymes carries out the conversion of arginine to citrulline while producing a molecule of nitric oxide (NO) in response to various stimuli. It is believed that excess nitric oxide (NO) production by NOS plays a role in the pathology of a number of disorders and conditions in mammals. For example, NO produced by I-NOS is thought to play a role in diseases that involve systemic hypotension such as toxic shock and therapy with certain cytokines. It has been shown that cancer patients treated with cytokines such as interleukin 1 (IL-1), interleukin 2 (IL-2) or tumor necrosis factor (TNF) suffer cytokine-induced shock and hypotension due to NO produced from macrophages, i.e., inducible NOS (I-NOS), see Chemical & Engineering News, December 20, p. 33, (1993). I-NOS inhibitors can reverse this. It is also believed that I-NOS plays a role in the pathology of diseases of the central nervous system such as ischemia. For example, inhibition of I-NOS has been shown to ameliorate cerebral ischemic damage in rats, see Am. J. Physiol., 268, p. R286 (1995)). Suppression of adjuvant induced arthritis by selective inhibition of I-NOS is reported in Eur. J. Pharmacol., 273, p. 15-24 (1995).

NO produced by N-NOS is thought to play a role in diseases such as cerebral ischemia, pain, and opiate tolerance. For example, inhibition of N-NOS decreases infarct volume after proximal middle cerebral artery occlusion in the rat, see J. Cerebr. Blood Flow Metab., 14, p. 924-929 (1994). N-NOS inhibition has also been shown to be effective in antinociception, as evidenced by activity in the late phase of the formalin-induced hindpaw licking and acetic acid-induced abdominal constriction assays, see Br. J. Pharmacol., 110, p. 219-224 (1993). In addition, subcutaneous injection of Freund's adjuvant in the rat induces an increase in NOS-positive neurons in the spinal cord that is manifested in increased sensitivity to pain, which can be treated with NOS inhibitors, see Japanese Journal of Pharmacology, 75, p. 327-335 (1997). Finally, opioid withdrawal in rodents has been reported to be reduced by N-NOS inhibition, see Neuropsychopharmacol., 13, p. 269-293 (1995).

### SUMMARY OF THE INVENTION

This invention also relates to a method of treating an inflammatory disorder such as rheumatoid arthristis, osteoarthritis, psoriasis or asthma in a mammal, including a human, comprising administering to said mammal:

(a) a NOS inhibiting compound, or pharmaceutically acceptable salt thereof; and

(b) a compound that exhibits antiinflammatory activity (such as sentanyl, morphine, or meperidine, or a steroidal antiinflammatory compound such as inhibitors of cyclooxygenase), or a pharmaceutically acceptable salt thereof;

wherein the active agents “a” and “b” above are present in amounts that render the combination of the two agents effective in treating such disorder.

This invention also relates to a method of treating chronic or acute pain in a mammal, including a human, comprising adminstering to said mammal:

(a) a NOS inhibiting compound or pharmaceutically acceptable salt thereof; and

(b) a narcotic analgesic compound (e.g., an opiate such as morphine or demerol), or a pharmaceutically acceptable salt thereof;

wherein the active agents “a” and “b” above are present in amounts that render the combination of the two agents effective in treating chronic or acute pain.

This invention also relates to a pharmaceutical composition for treating an inflammatory disorder (such as rheumatoid arthritis, osteoarthritis, psoriasis or asthma) in a mammal, including a human, comprising:

(a) a compound that exhibits antiinflammatory activity (such as sentanyl, morphine, or meperidine, or a steroidal antiinflammatory compound such as inhibitors of cyclooxygenase), or a pharmaceutically acceptable salt thereof;

(b) a NOS inhibiting compound or pharmaceutically acceptable salt thereof; and

(c) a pharmaceutically acceptable carrier;

wherein the active agents “a” and “b” are present in such composition in amounts that render the combination of the two agents effective in treating such disorder.

This invention also relates to a pharmaceutical composition for treating chronic or acute pain in a mammal, including a human, comprising:

(a) a NOS inhibiting compound or pharmaceutically acceptable salt thereof; and

(b) a narcotic analgesic compound (e.g., an opiate such as morphine or demerol) or a pharmaceutically acceptable salt thereof; and

(c) a pharmaceutically acceptable carrier;

wherein the active agents “a” and “b” are present in such composition in amounts that render the combination of the two agents effective in treating chronic or acute pain.

This invention also relates to a pharmaceutical composition for treating a condition selected from migraine, cluster and other vascular headaches in a mammal, including a human, comprising:

(a) a NOS inhibiting compound or pharmaceutically acceptable salt thereof; and

(b) a serotonin-1D (5HT_{1D}) receptor agonist (e.g., eletriptan or sumatriptan) or a pharmaceutically acceptable salt thereof; and

(c) a pharmaceutically acceptable carrier;

wherein the active agents “a” and “b” are present in such composition in amounts that render the combination of the two agents effective in treating such condition.

This invention also relates to a method for treating a condition selected from migraine, cluster and other vascular headaches in a mammal, including a human, comprising administering to said mammal:

(a) a NOS inhibiting compound or pharmaceutically acceptable salt thereof; and

(b) a serotonin-1D (5HT_{1D}) receptor agonist (e.g., eletriptan or sumatriptan) or a pharmaceutically acceptable salt thereof;

wherein the active agents “a” and “b” are present in such composition in amounts that render the combination of the two agents effective in treating such condition.

This invention also relates to any of the above methods wherein the NOS inhibiting compound is a compound of the formula I, II, III, IV, V or VI, as defined below.

The term “treating” as used herein, refers to reversing, alleviating, inhibiting the progress of, or preventing the disorder or condition to which such term applies, or one or more symptoms of such disorder or condition. The term “treatment”, as used herein, refers to the act of treating, as “treating” is defined immediately above.

This invention also relates to a pharmaceutical composition for treating a condition selected from the group consisting of sleep disorders, psoriasis and cognitive deficits or disorders in a mammal, including a human, comprising an amount of a NOS inhibiting compound of the formula I, II, III, IV, V or VI, as defined below, that is effective in treating such condition and a pharmaceutically acceptable carrier.

This invention also relates to a method of treating a condition selected from the group consisting of sleep disorders, psoriasis and cognitive deficits or disorders in a mammal, including a human, comprising administering to said mammal an amount of a NOS inhibiting compound of the formula I, II, III, IV, V or VI, as defined below, that is effective in treating or preventing such condition.

This invention also relates to a pharmaceutical composition for treating or preventing a condition selected from the group consisting of sleep disorders, psoriasis and cognitive deficits or disorders in a mammal, including a human, comprising a NOS inhibiting effective amount of a compound of the formula I, II, III, IV, V or VI, as defined below, or a pharmaceutically acceptable salt thereof, and a pharmaceutically acceptable carrier.

This invention also relates to a method of treating a condition selected from the group consisting of sleep disorders, psoriasis and cognitive deficits or disorders in a mammal, comprising administering to said mammal a NOS inhibiting effective amount of a compound of the formula I, II, III, IV, V or VI, as defined below, or a pharmaceutically acceptable salt thereof.

Examples of NOS inhibiting compounds that can be used in the methods and pharmaceutical compositions of the present invention are compounds of the formula

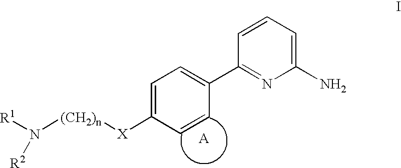

wherein ring A is a fused 5-7 membered saturated or unsaturated ring wherein from zero to two of the ring members are heteroatoms selected, independently, from nitrogen, oxygen and sulfur, with the proviso that no two adjecent ring members can both be heteroatoms;

X is oxygen or a bond;

n is an interger from two to six; and

R¹ and R² are selected, independently, from (C₁-C₆) alkyl, aryl, tetrahydronaphthalene and aralkyl, wherein said aryl and the aryl moiety of said aralkyl is phenyl or naphthyl and the alkyl moiety is straight or branched and contains from 1 to 6 carbon atoms, and wherein said (C₁-C6) alkyl, said aryl, said tetrahydronaphthalene and the aryl moiety of said aralkyl may optionally be substituted with from one to three substituents, preferably from zero to two substituents, that are selected, independently, from halo (e.g., chloro, fluoro, bromo, iodo), nitro, hydroxy, cyano, amino, (C₁-C₄) alkoxy, and (C₁-C₄) alkylamino;

or R¹ and R² form, together with the nitrogen to which they are attached, a piperazine, azetidine, piperidine or pyrrolidine ring or an azabicyclic ring containing from 6 to 14 ring members, from 1 to 3 of which are nitrogen and the rest of which are carbon, wherein examples of said azabicyclic rings are the following

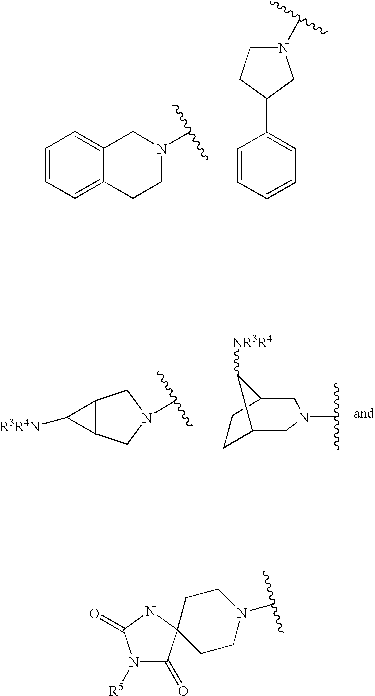

also R¹ or R² may be connected onto the (CH2)ₙ group to form a ring of from 4 to 7 members;

wherein R³ and R⁴ are selected from hydrogen, (C₁-C6)alkyl, phenyl, naphthyl, (C₁-C₆)alkyl-C(═O)—, HC(═O)—, (C₁-C₆)alkoxy-(C═O)—, phenyl-C(═O)—, naphthyl-C(═O)—, and R⁶R⁷NC(═O)— wherein R⁶ and R⁷ are selected, independently, from hydrogen and (C₁-C₆)alkyl;

R⁵ is selected from hydrogen, (C₁-C₆)alkyl, phenyl, naphthyl, phenyl-(C₁-C₆)alkyl- and naphthyl(C₁-C₆)alkyl-;

and wherein said piperazine, azetidine, piperidine and pyrrolidine rings may optionally be substituted with one or more substituents, preferably with from zero to two substituents that are selected, independently, from (C₁-C₆)alkyl, amino, (C₁-C₆) alkylamino, [di-(C₁-C₆)alkyl]amino, phenyl substituted 5 to 6 membered heterocyclic rings containing from 1 to 4 rings nitrogen atoms, benzoyl, benzoylmethyl, benzylcarbonyl, phenylaminocarbonyl, phenylethyl and phenoxycarbonyl, and wherein the phenyl moieties of any of the foregoing substituents may optionally be substituted with one or more substituents, preferably with from zero to two substituents, that are selected, independently, from halo, (C₁-C₃)alkyl, (C₁-C₃)alkoxy, nitro, amino, cyano, CF₃ and OCF₃;

and the pharmaceutically acceptable salts of such compounds.

The following compounds are preferred NOS inhibitors of the formula l:

6-[4-(2-Dimethylamino-ethoxy)-naphthalen-1-yl]-pyridin-2-ylamine;

6-[4-(2-Pyrrolidin-1-yl-ethoxy)-naphthalen-1-yl]-pyridin-2-ylamine;

6-(4-{2-[(Benzo[1,3]dioxol-5-ylmethyl)-amino]-ethoxy}-naphthalen-1-yl)-pyridin-2-ylamine;

6-{4-[2-(6,7-Dimethoxy-3,4-dihydro-1H-isoquinolin-2-yl)-ethoxy]-naphthalen-1-yl)-pyridin-2-ylamine;

3-{2-[4-(6-Amino-pyridin-2-yl)-naphthalen-1-yloxy]-ethyl}-3-aza-bicyclo[3.1.0]hex-6-ylamine;

6-{4-[2-(4-Phenethyl-piperazin-1-yl)-ethoxy]-naphthalen-1-yl}-pyridin-2-ylamine;

6-{4-[2-(3-Amino-pyrrolidin-1-yl)-ethoxy]-naphthalen-1-yl}-pyridin-2-ylamine;

6-[4-(1-Benzyl-piperidin-4-yloxy)-naphthalen-1-yl]-pyridin-2-ylamine;

6-[4-(1-Benzyl-pyrrolidin-3-yloxy)-naphthalen-1-yl]-pyridin-2-ylamine;

6-[4-(Piperidin-4-yloxy)-naphthalen-1-yl]-pyridin-2-ylamine;

6-[4-(Pyrrolidin-3-yloxy)-naphthalen-1-yl]-pyridin-2-ylamine;

6-[4-(1-lsobutyl-piperidin-4-yloxy)-naphthalen-1-yl]-pyridin-2-ylamine;

6-[4-(1-Furan-2-ylmethyl-piperidin-4-yloxy)-naphthalen-1-yl]-pyridin-2-ylamine;

6-[4-(1-Isobutyl-pyrrolidin-3-yloxy)-naphthalen-1-yl]-pyridin-2-ylamine;

6-[4-(1-Furan-2-ylmethyl-pyrrolidin-3-yloxy)-naphthalen-1-yl]-pyridin-2-ylamine;

6-[4-(2-Morpholin-4-yl-ethoxy)-naphthalen-1-yl]-pyridin-2-ylamine;

6-[4-(2-Diisopropylamino-ethoxy)-naphthalen-1-yl]-pyridin-2-ylamine;

6-[4-(1-Methyl-piperidin-4-yloxy)-naphthalen-1-yl]-pyridin-2-ylamine;

6-[4-(1-Methyl-pyrrolidin-3-yloxy)-naphthalen-1-yl]-pyridin-2-ylamine;

6-[4-(3-Dimethylamino-propoxy)-naphthalen-1-yl]-pyridin-2-ylamine;

6-[4-(1-Aza-bicyclo[2.2.2]oct-3-yloxy)-naphthalen-1-yl]-pyridin-2-ylamine;

6-[4-(2-Piperidin-1-yl-ethoxy)-naphthalen-1-yl]-pyridin-2-ylamine

6-{4-[2-(3,4-Dihydro-1H-isoquinolin-2-yl)-ethoxy]-naphthalen-1-yl}-pyridin-2-ylamine;

6-{4-[2-(4-Dimethylamino-piperidin-1-yl)-ethoxy]-naphthalen-1-yl}-pyridin-2-ylamine;

6-{4-[2-(tert-Butyl-methyl-amino)-ethoxy]-naphthalen-1-yl}-pyridin-2-ylamine;

6-{4-[2-(4-Methyl-piperazin-1-yl)-ethoxy]-naphthalen-1-yl}-pyridin-2-ylamine;

6-{4-[2-(4-Phenyl-piperidin-1-yl)-ethoxy]-naphthalen-1-yl}-pyridin-2-ylamine;

6-{4-[2-(7,8-Dihydro-5H-[1,3]dioxolo[4,5-g]isoquinolin-6-yl)-ethoxy]-naphthalen-1-yl}-pyridin-2-ylamine;

6-[4-(Piperidin-2-ylmethoxy)-naphthalen-1-yl]-pyridin-2-ylamine;

6-[4-(1-Methyl-piperidin-2-ylmethoxy)-naphthalen-1-yl]-pyridin-2-ylamine;

6-[4-(1-Methyl-piperidin-3-ylmethoxy)-naphthalen-1-yl]-pyridin-2-ylamine;

6-[4-(2-Amino-cyclohexyloxy)-naphthalen-1-yl]-pyridin-2-ylamine;

6-[4-(Piperidin-3-ylmethoxy)-naphthalen-1-yl]-pyridin-2-ylamine;

6-[4-(1-Isobutyl-azetidin-3-yloxy)-naphthalen-1-yl]-pyridin-2-ylamine;

6-[4-(1-Furan-2-ylmethyl-azetidin-3-yloxy)-naphthalen-1-yl]-pyridin-2-ylamine;

6-[4-(8-Methyl-8-aza-bicyclo[3.2.1]oct-3-yloxy)-naphthalen-1-yl]-pyridin-2-ylamine,

6-[4-(Azetidin-3-yloxy)-naphthalen-1-yl]-pyridin-2-ylamine;

6-[4-(1-Methyl-pyrrolidin-2-ylmethoxy)-naphthalen-1-yl]-pyridin-2-ylamine;

6-[4-(Azetidin-2-ylmethoxy)-naphthalen-1-yl]-pyridin-2-ylamine;

6-[7-(2-Dimethylamino-ethoxy)-indan-4-yl]-pyridin-2-ylamine;

6-[7-(2-Pyrroidin-1-yl-ethoxy)-indan-4-yl]-pyridin-2-ylamine;

6-{7-[2-Benzyl-methyl-amino)-ethoxy]-indan-4-yl}-pyridin-2-ylamine;

6-{7-[2-(4-Phenethyl-piperazin-1-yl)-ethoxy]-indan-4-yl}-pyridin-2-ylamine;

6-{7-[2-(4-Isobutyl-piperazin-1-yl)-ethoxy]-indan-4-yl}-pyridin-2-ylamine;

6-[7-(2-Morpholin-4-yl-ethoxy)-indan-4-yl]-pyridin-2-ylamine;

6-[7-(2-Diisopropylamino-ethoxy)-indan-4-yl]-pyridin-2-ylamine;

6-{7-[2-(7,8-Dihydro-5H-[1,3]dioxolo[4,5-g]isoquinolin-6-yl)-ethoxy]-indan-4-yl}-pyridin-2-ylamine;

6-{7-[2-(4-Methyl-piperazin-1-yl)-ethoxy]-indan-4-yl}-pyridin-2-ylamine;

6-{7-[2-(tert-Butyl-methyl-amino)-ethoxy]-indan-4-yl)-pyridin-2-ylamine;

6-{7-[2-(4-Dimethylamino-piperidin-1-yl)-ethoxy]-indan-4-yl}-pyridin-2-ylamine;

6-[8-(2-Dimethylamino-ethoxy)-1,2,3,4-tetrahydro-1,4-methano-naphthalen-5-yl]-pyridin-2-ylamine;

6-[8-(2-Pyrrolidin-1-yl-ethoxy)-1,2,3,4-tetrahydro-1,4-methano-naphthalen-5-yl]-pyridin-2-ylamine;

6-[4-(2-Dimethylamino-ethoxy)-5,6,7,8-tetrahydro-naphthalen-1-yl]-pyridin-2-ylamine;

6-[4-(2-Pyrrolidin-1-yl-ethoxy)-5,6,7,8-tetrahydro-naphthalen-1-yl]-pyridin-2-ylamine;

6-{4-[2-(tert-Butyl-methyl-amino)-ethoxy]-5,6,7,8-tetrahydro-naphthalen-1-yl}-pyridin-2-ylamine;

6-[4-(2-Diisopropylamino-ethoxy)-5,6,7,8-tetrahydro-naphthalen-1-yl]-pyridin-2-ylamine;

6-[4-(2-Diethylamino-ethoxy)-5,6,7,8-tetrahydro-naphthalen-1-yl]-pyridin-2-ylamine;

6-{4-[2-(3,4-Dihydro-1H-isoquinolin-2-yl)-ethoxy]-5,6,7,8-tetrahydro-naphthalen-1-yl)}-pyridin-2-ylamine;

6-[4-(2-Piperidin-1-yl-ethoxy)-5,6,7,8-tetrahydro-naphthalen-1-yl]-pyridin-2-ylamine;

6-[4-(2-Morpholin-4-yl-ethoxy)-5,6,7,8-tetrahydro-naphthalen-1-yl]-pyridin-2-ylamine;

6-{4-[2-(7,8-Dihydro-5H-[1,3]dioxolo[4,5-g]isoquinolin-6-yl)-ethoxy]-5,6,7,8-tetrahydro-naphthalen-1-yl}-pyridin-2-ylamine;

6-{4-[2-(4-Methyl-piperazin-1-yl)-ethoxy]-5,6,7,8-tetrahydro-naphthalen-1-yl}-pyridin-2-ylamine;

6-({4-[2-(4-Dimethylamino-piperidin-1-yl)-ethoxy]-5,6,7,8-tetrahydro-naphthalen-1-yl}-pyridin-2-ylamine;

6-{4-[2-(7,8-Dihydro-5H-[1,3]dioxolo[4,5-g]isoquinolin-6-yl)-ethoxy]-5,6,7,8-tetrahydro-naphthalen-1-yl}-pyridin-2-ylamine;

6-[4-(1-Isobutyl-piperidin-3-ylmethoxy)-5,6,7,8-tetrahydro-naphthalen-1-yl]-pyridin-2-ylamine;

6-[4-(1-Methyl-piperidin-3-ylmethoxy)-5,6,7,8-tetrahydro-naphthalen-1-yl]-pyridin-2-ylamine;

6-{4-[2-(2-Diethylamino-ethoxy)-ethoxy]-5,6,7,8-tetrahydro-naphthalen-1-yl}-pyridin-2-ylamine;

6-[4-(Piperidin-3-ylmethoxy)-5,6,7,8-tetrahydro-naphthalen-1-yl]-pyridin-2-ylamine;

6-[4-(2-Amino-cyclohexyloxy)-5,6,7,8-tetrahydro-naphthalen-1-yl]-pyridin-2-ylamine;

6-[4-(Pyrrolidin-2-ylmethoxy)-5,6,7,8-tetrahydro-naphthalen-1-yl]-pyridin-2-ylamine; and

6-[4-(2-Dimethylamino-ethoxy)-6,7,8,9-tetrahydro-5H-benzocyclohepten-1-yl]-pyridin-2-ylamine;

and the pharmaceutically acceptable salts of the foregoing compounds.

The following are additional examples of NOS inhibiting compounds of the formula I.

6-[4-(2-Amino-cyclopentyloxy)-5,6,7,8-tetrahydro-naphthalen-1-yl]-pyridin-2-ylamine;

6-[4-(2-Amino-cyclobutyloxy)-5,6,7,8-tetrahydro-naphthalen-1-yl]-pyridin-2-ylamine;

6-[4-(2-Amino-cyclopropyloxy)-5,6,7,8-tetrahydro-naphthalen-1-yl]-pyridin-2-ylamine;

6-[4-(3-Amino-cyclohexyloxy)-5,6,7,8-tetrahydro-naphthalen-1-yl]-pyridin-2-ylamine;

6-[4-(3-Amino-cyclopenytloxy)-5,6,7,8-tetrahydro-naphthalen-1-yl]-pyridin-2-ylamine;

6-[4-(3-Amino-cyclobutyloxy)-5,6,7,8-tetrahydro-naphthalen-1-yl]-pyridin-2-ylamine;

6-[4-(4-Amino-cyclohexyloxy)-5,6,7,8-tetrahydro-naphthalen-1-yl]-pyridin-2-ylamine;

6-[4-(2-Amino-cyclopentyloxy)-naphthalen-1-yl]-pyridin-2-ylamine;

6-[4-(2-Amino-cyclobutyloxy)-naphthalen-1-yl]-pyridin-2-ylamine;

6-[4-(2-Amino-cyclopropyloxy)-naphthalen-1-yl]-pyridin-2-ylamine;

6-[4-(3-Amino-cyclohexyloxy)-naphthalen-1-yl]-pyridin-2-ylamine;

6-[4-(3-Amino-cyclopentyloxy)-naphthalen-1-yl]-pyridin-2-ylamine;

6-[4-(3-Amino-cyclobutyloxy)-naphthalen-1-yl]-pyridin-2-ylamine;

6-[4-(4-Amino-cyclohexyloxy)-naphthalen-1-yl]-pyridin-2-ylamine;

6-[4-(2-Amino-cyclopentyloxy)-indan-4-yl]-pyridin-2-ylamine;

6-[4-(2-Amino-cyclobutyloxy)-indan-4-yl]-pyridin-2-ylamine;

6-[4-(2-Amino-cyclopropyloxy)-indan-4-yl]-pyridin-2-ylamine;

6-[4-(3-Amino-cyclohexyloxy)-indan-4-yl]-pyridin-2-ylamine;

6-[4-(3-Amino-cyclopentyloxy)-indan-4-yl]-pyridin-2-ylamine;

6-[4-(3-Amino-cyclobutyloxy)-indan-4-yl]-pyridin-2-ylamine;

6-[4-(4-Amino-cyclohexyloxy)-indan-4-yl]-pyridin-2-ylamine;

6-[4-Piperidin-3-ylmethoxy)-6,7,8,9-tetrahydro-5H-benzoyclohepten-1-yl]-pyridin-2-ylamine;

6-[4-(2-Pyrrolidinyl-ethoxy)-6,7,8,9-tetrahydro-5H-benzocyclohepten-1-yl]-pyridin-2-ylamine;

6-[4-(2-Amino-cyclohexyloxy)-6,7,8,9-tetrahydro-5H-benzocyclohepten-1-yl]-pyridin-2-ylamine;

6-[4-(2-(4-Dimethylamino-piperidin-1-yl)-ethoxy))-6,7,8,9-tetrahydro-5H-benzocyclohepten-1-yl]-pyridin-2-ylamine; and

6-[4-(2-(4-Methyl-piperazin-1-yl)-ethoxy))-6,7,8,9-tetrahydro-5H-benzocyclohepten-1-yl]-pyridin-2-ylamine.

Other examples of NOS inhibiting compounds that can be used in the methods and pharmaceutical compositions of this invention are compounds of the formula

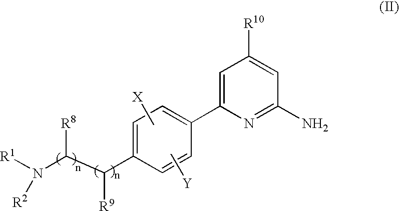

and the pharmaceutically acceptable salts thereof, wherein

R¹ and R² are selected, independently, from (C₁-C₆) alkyl, tetrahydronaphthalene and aralkyl, wherein the aryl moiety of said aralkyl is phenyl or naphthyl and the alkyl moiety is straight or branched and contains from 1 to 6 carbon atoms, and wherein said (C₁-C₆) alkyl and said tetrahydronaphthalene and the aryl moiety of said aralkyl may optionally be substituted with from one to three substituents, preferably from zero to two substituents, that are selected, independently, from halo (e.g., chloro, fluoro, bromo, iodo), nitro, hydroxy, cyano, amino, (C₁-C₄) alkoxy, and (C₁-C₄) alkylamino;

or R¹ and R² form, together with the nitrogen to which they are attached, a piperazine, piperidine or pyrrolidine ring or an azabicyclic ring containing from 6 to 14 ring members, from 1 to 3 of which are nitrogen and the rest of which are carbon, wherein examples of said azabicyclic rings are the following

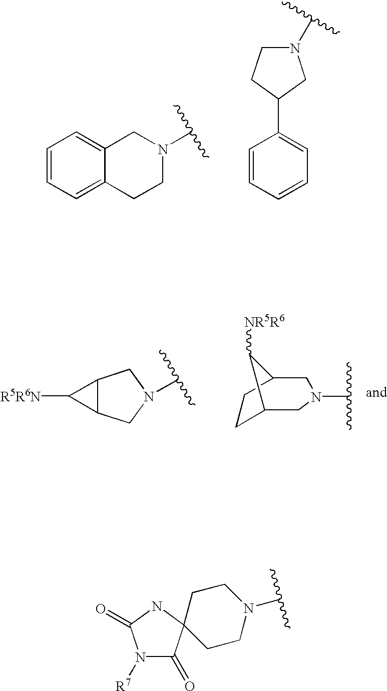

wherein R³ and R⁴ are selected from hydrogen, (C₁-C₆)alkyl, phenyl, naphthyl, (C₁-C₆)alkyl-C(═O)—, HC(═O)—, (C₁-C₆)alkoxy-(C═O)—, phenyl-C(═O)—, naphthyl-C(═O)—, and R⁷R⁸NC(═O)— wherein R⁷ and R⁸ are selected, independently, from hydrogen and (C₁-C₆)alkyl;

R⁵ is selected from hydrogen, (C₁-C₆)alkyl, phenyl, naphthyl, phenyl-(C₁-C₆)alkyl- and naphthyl(C₁-C₆)alkyl-;

and wherein said piperazine, piperidine and pyrrolidine rings may optionally be substituted with one or more substituents, preferably with from zero to two substituents that are selected, independently, from (C₁-C₆)alkyl, amino, (C₁-C₆) alkylamino, [di-(C₁-C₆)alkyl]amino, phenyl substituted 5 to 6 membered heterocyclic rings containing from 1 to 4 rings nitrogen atoms, benzoyl, benzoylmethyl, benzylcarbonyl, phenylaminocarbonyl, phenylethyl and phenoxycarbonyl, and wherein the phenyl moieties of any of the foregoing substituents may optionally be substituted with one or more substituents, preferably with from zero to two substituents, that are selected, independently, from halo, (C₁-C₃)alkyl, (C₁-C₃)alkoxy, nitro, amino, cyano, CF₃ and OCF₃;

n is 0,1 or 2;

m is 0,1, or 2;

each R⁸ and each R⁹ is selected, independently, from (C₁-C₄)alkyl, aryl-(C₁-C₄)alkyl wherein said aryl is selected from phenyl and naphthyl; allyl and phenallyl;

X and Y are selected, independently, from methyl, methoxy, hydroxy and hydrogen; and

R¹⁰ is (C₁-C₆) alkyl;

with the proviso that R⁸ is absent when N is zero and R⁹ is absent when m is zero.

Examples of preferred compounds of the formula II are those wherein NR¹R² is:

4-phenoxycarbonylpiperazin-1-yl;

4-(4-fluorophenylacetyl)piperazin-1-yl;

4-phenylethylpiperazin-1-yl;

4-phenoxymethylcarbonylpiperazin-1-yl;

4-phenylaminocarbonylpiperazin-1-yl;

4-benzoylmethylpiperazin-1-yl; or

4-benzylcarbonylpiperazin-1-yl.

Other preferred compounds of the formula II are those wherein NR¹R² is a group of the formula

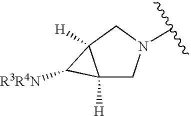

wherein NR³R⁴ is NH₂.

Other preferred compounds of the formula II are those wherein NR¹R² is a group of the formula

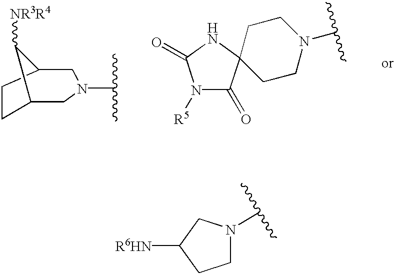

wherein R⁵ is aralkyl, e.g., benzyl, and R⁶ is (4-fluoro)phenylacetyl.

Specific preferred compounds of the formula II include:

1-(4-{2-[4-(6-Amino-pyridin-2-yl)-phenyl]-ethyl}-piperazin-1-yl)-ethanone;

1-(4-{2-[4-(6-Amino-pyridin-2-yl)-phenyl]-ethyl}-piperazin-1-yl)-2-methoxy-ethanone;

1-(4-{2-[4-(6-Amino-pyridin-2-yl)-phenyl]-ethyl}-piperazin-1-yl)-2-phenoxy-ethanone;

(4-{2-[4-(6-Amino-pyridin-2-yl)-phenyl]-ethyl}-piperazin-1-yl)-cyclopentyl-methanone;

1-(4-{2-[4-(6-Amino-pyridin-2-yl)-phenyl]-ethyl}-piperazin-1-yl)-2-phenyl-ethanone;

3-{2-[4-(6-Amino-pyridin-2-yl)-phenyl]-ethyl}-3-aza-bicyclo[3.1.0]hex-6-ylamine;

2-(4-{2-[4-(6-Amino-pyridin-2-yl)-phenyl]-ethyl}-piperazin-1-yl)-1-phenyl-ethanone;

1-(4-{2-[4-(6-Amino-pyridin-2-yl)phenyl]-ethyl}-piperazin-1-yl)-2-(4-fluoro-phenyl)-ethanone;

6-{4-[2-(4-Phenethyl-piperazin-1-yl)-ethyl]-phenyl}-pyridin-2-ylamine;

2-(4-{2-[4-(6-Amino-pyridin-2-yl)-phenyl]-ethyl}-piperazin-1-yl)-1-phenyl-ethanol;

{2-[4-(6-Amino-pyridin-2-yl)-phenyl]-ethyl}-(3-oxa-9-aza-bicyclo[3.3.1]non-7-yl)-amine;

6-(4-{2-[4-(2-Amino-2-phenyl-ethyl)-piperazin-1-yl]-ethyl}-phenyl)-pyridin-2-ylamine;

6-{4-[2-(4-Amino-2,6-dimethyl-piperidin-1-yl)-ethyl]-phenyl)}-pyridin-2-ylamine;

6-{4-[2-(4-Methyl-piperazin-1-yl)-ethyl]-phenyl}-pyridin-2-ylamine;

(3-{2-[4-(6-Amino-pyridin-2-yl)-phenyl]-ethyl}-3-aza-bicyclo[3.1.0]hex-6-yl)-dimethyl-amine;

6-[4-(2-Amino-ethyl)-phenyl]-pyridin-2-ylamine;

6-{4-[2-(8-Aza-spiro[4.5]dec-8-yl)-ethyl]-phenyl}-pyridin-2-ylamine;

6-{4-[2-(4-Isobutyl-piperazin-1-yl)-ethyl]-phenyl}-pyridin-2-ylamine;

2-(4-{2-[4-(6-Amino-pyridin-2-yl)-phenyl]-ethyl}-piperazin-1-yl)-N-isopropyl-acetamide;

4-{2-[4-(6-Amino-pyridin-2-yl)-phenyl]-ethyl}-piperazine-1-carboxylic acid p-tolyl-amide;

6-(4-{2-[4-(3-Phenyl-propyl)-piperazin-1-yl]-ethyl}-phenyl)-pyridin-2-ylamine;

1-(4-{2-[4-(6-Amino-pyridin-2-yl)-phenyl]-ethyl}-piperazin-1-yl)-2-(4-chloro-phenyl)-ethanone;

8-{2-[4-(6-Amino-pyridin-2-yl)-phenyl]-ethyl}-3-benzyl-1,3,8-triaza-spiro[4.5]decane 2,4-dione;

N-(1-{2-[4-(6-Amino-pyridin-2-yl)-phenyl]-ethyl}-pyrrolidin-3-yl)-2-(4-fluoro-phenyl)-acetamide;

8-{2-[4-(6-Amino-pyridin-2-yl)-phenyl]-ethyl}-8-aza-bicyclo[3.2.1]oct-3-ylamine;

3-{2-[4-(6-Amino-pyridin-2-yl)-phenyl]-ethyl}-3-aza-bicyclo[3.2.1]oct-8-ylamine;

2-Amino-1-(4-{2-[4-(6-amino-pyridin-2-yl)-phenyl]-ethyl}-piperazin-1-yl)-3-phenyl-propan-1-one;

6-{4-[2-(4-Amino-piperidin-1-yl)-ethyl]-phenyl}-pyridin-2-ylamine;

6-{4-[2-(4-Benzhydryl-piperazin-1-yl)-ethyl]-phenyl}-pyridin-2-ylamine;

6-{4-[2-(4-Benzhydryl-piperidin-1-yl)-ethyl]-phenyl}-pyridin-2-ylamine;

6-{4-[(Cyclohexyl-methyl-amino)-methyl]-phenyl}-pyridin-2-ylamine;

6-{4-[(Cyclohexyl-methyl-amino)-methyl]-2-methoxy-phenyl}-pyridin-2-ylamine;

6-[4-(Phenethylamino-methyl)-phenyl]-pyridin-2-ylamine;

6-[2-Methoxy-4-(phenethylamino-methyl)-phenyl]-pyridin-2-ylamine;

6-[4-(4-Amino-piperidin-1-ylmethyl)-phenyl}-pyridin-2-ylamine;

6-{4-[(Cyclohexyl-methyl-amino)-methyl]-2-fluoro-phenyl}-pyridin-2-ylamine;

Other compounds of the formula II include:

1-(4-{2-[4-(6-Amino-pyridin-2-yl)-2-methoxy-phenyl]-ethyl}-piperazin-1-yl)-2-ethanone;

6-{4-[2-(4-Isobutyl-piperazin-1-yl)-ethyl]-2-methoxy-phenyl}-pyridin-2-ylamine;

3-{2-[4-(6-Amino-pyridin-2-yl)-2-methoxy-phenyl]-ethyl}-3-aza-bicyclo[3.1.0]hex-6-ylamine;

{2-[4-(6-Amino-pyridin-2-yl)-2-methoxy-phenyl]-ethyl}-(3-oxa-9-aza-bicyclo[3.3.1]non-7-yl)-amine;

6-(4-{2-[4-(2-Amino-2-phenyl-ethyl)-piperazin-1-yl]-ethyl}-2-methoxy-phenyl)-pyridin-2-ylamine;

6-{4-[2-(4-Amino-2-methoxy-piperidin-1-yl)-ethyl]-2-methoxy-phenyl}-pyridin-2-ylamine;

2-(4-{2-[4-(6-Amino-pyridin-2-yl)-2-methoxy-phenyl]-ethyl}-piperazin-1-yl)-N-isopropyl-acetamide;

6-[4-(4-Amino-piperidin-1-ylmethyl)-2-methoxy-phenyl}-pyridin-2-ylamine;

1-(4-{2-[4-(6-Amino-pyridin-2-yl)-2-methyl-phenyl]-ethyl}-piperazin-1-yl)-2-phenyl-ethanone;

6-{4-[2-(4-Isobutyl-piperazin-1-yl)-ethyl]-2-methyl-phenyl}-pyridin-2-ylamine;

3-}2-[4-(6-Amino-pyridin-2-yl)-2-methyl-phenyl]-ethyl}-3-aza-bicyclo[3.1.0]hex-6-ylamine;

2-(4-{2-[4-(6-Amino-pyridin-2-yl)-2-methyl-phenyl]-ethyl}-piperazin-1-yl)-1-phenyl-ethanone;

1-(4-{2-[4-(6-Amino-pyridin-2-yl)-2-methyl-phenyl]-ethyl}-piperazin-1-yl)-2-(4-fluoro-phenyl)-ethanone;

6-{4-[2-(4-Phenethyl-piperazin-1-yl)-ethyl]-2-methyl-phenyl}-pyridin-2-ylamine;

2-(4-{2-[4-(6-Amino-pyridin-2-yl)-2-methyl-phenyl]-ethyl}-piperazin-1-yl)-1-phenyl-ethanol;

{2-[4-(6-Amino-pyridin-2-yl)-2-methyl-phenyl]-ethyl}-(3-oxa-9-aza-bicyclo[3.3.1]non-7-yl)-amine;

6-(4-{2-[4-(2-Amino-2-phenyl-ethyl)-piperazin-1-yl]-ethyl}-2-methyl-phenyl)-pyridin-2-ylamine;

6-{4-[2-(4-Amino-2,6-dimethyl-piperidin-1-yl)-ethyl]-2-methyl-phenyl}-pyridin-2-ylamine;

2-(4-{2-[4-(6-Amino-pyridin-2-yl)-2-methyl-phenyl]-ethyl}-piperazin-1-yl)-N-isopropyl-acetamide;

6-[4-(4-Amino-piperidin-1-ylmethyl)-2-methyl-phenyl}-pyridin-2-ylamine;

N-(1-{2-[4-(6-Amino-pyridin-2-yl)-phenyl]-ethyl}-pyrrolidin-3-yl)-2-phenyl-acetamide;

N-(1-{2-[4-(6-Amino-pyridin-2-yl)-phenyl]-ethyl}-pyrrolidin-3-yl)-2-(3-trifluoromethylphenyl)-acetamide;

N-(1-{2-[4-(6-Amino-pyridin-2-yl)-phenyl]-ethyl}-pyrrolidin-3-yl)-2-(4-tolyl)-acetamide;

N-(1-{2-[4-(6-Amino-pyridin-2-yl)-phenyl]-ethyl}-pyrrolidin-3-yl)-2-(4-methoxyphenyl)-acetamide;

2-(4-{2-[4-(6-Amino-pyridin-2-yl)-2-methoxy-phenyl]-ethyl}-piperazin-1-yl)-1-phenyl-ethanone;

1-(4-{2-[4-(6-Amino-pyridin-2-yl)-2-methoxy-phenyl]-ethyl}-piperazin-1-yl)-2-(4-fluoro-phenyl)-ethanone;

N-(1-{2-[4-(6-Amino-pyridin-2-yl)-phenyl]-ethyl}-pyrrolidin-3-yl)-2-cyclohexyl-acetamide;

2-(4-{2-[4-(6-Amino-pyridin-2-yl)-phenyl]-ethyl}-piperazin-1-yl)-1-(4-tolyl)-ethnone;

2-(4-{2-[4-(6-Amino-pyridin-2-yl)-phenyl]-ethyl}-piperazin-1-yl)-1-(4-methoxyphenyl)-ethanone;

2-(4-{2-[4-(6-Amino-pyridin-2-yl)-phenyl]-ethyl}-piperazin-1-yl)-1-(4-chlorophenyl)-ethanone;

2-(4-{2-[4-(6-Amino-pyridin-2-yl)-phenyl]-ethyl}-piperazin-1-yl)-1-(4-fluorophenyl)-ethanone;

2-(4-{2-[4-(6-Amino-pyridin-2-yl)-phenyl]-ethyl}-piperazin-1-yl)-1-cyclohexyl-ethanone;

1-(4-{2-[4-(6-Amino-pyridin-2-yl)-2-fluoro-phenyl]-ethyl}-piperazin-1-yl)-2-phenyl-ethanone;

6-{4-[2-(4-Isobutyl-piperazin-1-yl)-ethyl]-2-fluoro-phenyl}-pyridin-2-ylamine;

3-{2-[4-(6-Amino-pyridin-2-yl)-2-fluoro-phenyl]-ethyl}-3-aza-bicyclo[3.1.0]hex-6-ylamine;

2-(4-{2-[4-(6-Amino-pyridin-2-yl)-2-fluoro-phenyl]-ethyl}-piperazin-1-yl)-1-phenyl-ethanone;

1-(4-{2-[4-(6-Amino-pyridin-2-yl)-2-fluoro-phenyl]-ethyl}-piperazin-1-yl)-2-(4-fluoro-phenyl)-ethanone;

6-{4-[2-(4-Phenethyl-piperazin-1-yl)-ethyl]-2-fluoro-phenyl}-pyridin-2-ylamine;

2-(4-{2-[4-(6-Amino-pyridin-2-yl)-2-fluoro-phenyl]-ethyl}-piperazin-1-yl)-1-phenyl-ethanol;

{2-[4-(6-Amino-pyridin-2-yl)-2-fluoro-phenyl]-ethyl}-(3-oxa-9-aza-bicyclo[3.3.1]non-7-yl)-amine;

6-(4-{2-[4-(2-Amino-2-phenyl-ethyl)-piperazin-1-yl]-ethyl}-2-fluoro-phenyl)-pyridin-2-ylamine;

6-{4-[2-(4-Amino-2-fluoro-piperidin-1-yl)-ethyl]-2-fluoro-phenyl}-pyridin-2-ylamine;

2-(4-{2-[4-(6-Amino-pyridin-2-yl)-2-fluoro-phenyl]-ethyl}-piperazin-1-yl)-N-isopropyl-acetamide;

6-[4-(4-Amino-piperidin-1-ylmethyl)-2-fluoro-phenyl}-pyridin-2-ylamine;

6-{4-[2-(4-Amino-2,6-diethyl-piperidin-1-yl)-ethyl]-phenyl}-pyridin-2-ylamine;

6-{4-[2-(4-Amino-2,6-dibenzyl-piperidin-1-yl)-ethyl]-phenyl}-pyridin-2-ylamine;

{2-[4-(6-Amino-pyridin-2-yl)-phenyl]-ethyl}-(9-(4-fluoro)-benzyl-3-oxa-9-aza-bicyclo[3.3.1]non-7-yl)-amine;

{2-[4-(6-Amino-pyridin-2-yl)-phenyl]-ethyl}-(9-(4-chloro)-benzyl-3-oxa-9-aza-bicyclo[3.3.1]non-7-yl)-amine;

{2-[4-(6-Amino-pyridin-2-yl)-phenyl]-ethyl}-(9-(4-methyl)-benzyl-3-oxa-9-aza-bicyclo[3.3.1]non-7-yl)-amine; and

{2-[4-(6-Amino-pyridin-2-yl)-phenyl]-ethyl}-(9-(4-methoxy)-benzyl-3-oxa-9-aza-bicyclo[3.3.1]non-7-yl)-amine.

Other examples of NOS inhibiting compounds that can be used in the methods and pharmaceutical compositions of this invention are compounds of the formula

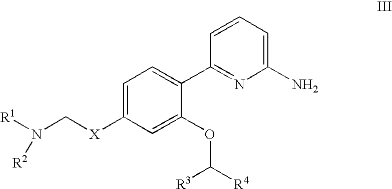

wherein X is CHOH, CH₂, or CHR¹⁰ wherein R¹⁰, together with X, the CH₂ group adjacent to X and the nitrogen of NR¹R², forms a five or six membered saturated ring;

R¹, R², R³ and R⁴ are selected, independently, from (C₁-C₆) alkyl, tetrahydronaphthalene, aryl and aralkyl, wherein said aryl and the aryl moiety of said aralkyl is phenyl or naphthyl and the alkyl moiety is straight or branched and contains from 1 to 6 carbon atoms, and wherein said (C₁-C₆) alkyl and said tetrahydronaphthalene and the aryl moiety of said aralkyl may optionally be substituted with from one to three substituents, preferably from zero to two substituents, that are selected, independently, from halo (e.g., chloro, fluoro, bromo, iodo), nitro, hydroxy, cyano, amino, (C₁-C₄) alkoxy, and (C₁-C₄) alkylamino;

or R¹ and R², together with the nitrogen to which they are attached, form a piperazine, piperidine or pyrrolidine ring or an azabicyclic ring containing from 6 to 14 ring members, from 1 to 3 of which are nitrogen and the rest of which are carbon, wherein examples of said azabicyclic rings are the following

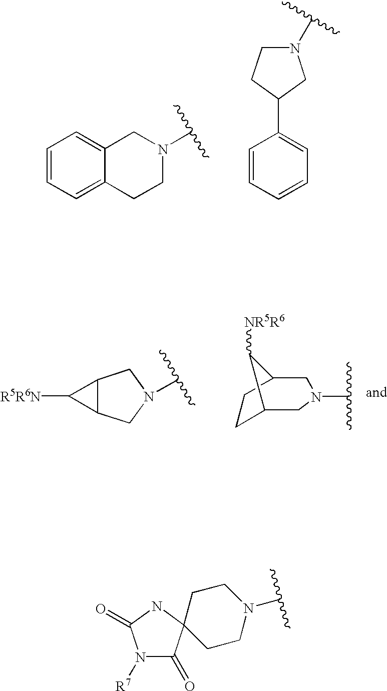

wherein R⁵ and R⁶ are selected from hydrogen, (C₁-C₆)alkyl, phenyl, naphthyl, (C₁-C₆)alkyl-C(═O)—, HC(═O)—, (C₁-C₆)alkoxy-(C═O)—, phenyl-C(═O)—, naphthyl-C(═O)—, and R⁸R⁹NC(═O)— wherein R⁸ and R⁹ are selected, independently, from hydrogen and (C₁-C₆)alkyl;

R⁷ is selected from hydrogen, (C₁-C₆)alkyl, phenyl, naphthyl, phenyl-(C₁-C₆)alkyl- and naphthyl(C₁-C₆)alkyl-;

and wherein said piperazine, piperidine and pyrrolidine rings may optionally be substituted with one or more substituents, preferably with from zero to two substituents that are selected, independently, from (C₁-C₆)alkyl, amino, (C₁-C₆) alkylamino, [di-(C₁-C₆)alkyl]amino, phenyl substituted 5 to 6 membered heterocyclic rings containing from 1 to 4 rings nitrogen atoms, benzoyl, benzoylmethyl, benzylcarbonyl, phenylaminocarbonyl, phenylethyl and phenoxycarbonyl, and wherein the phenyl moieties of any of the foregoing substituents may optionally be substituted with one or more substituents, preferably with from zero to two substituents, that are selected, independently, from halo, (C₁-C₃)alkyl, (C₁-C₃)alkoxy, nitro, amino, cyano, CF₃ and OCF₃;

and wherein R³ and R⁴, together with the carbon to which they are attached, form an optionally substituted carbocyclic ring of from 3 to 8 members;

and the pharmaceutically acceptable salts of such compounds.

More specific embodiments of compounds of the formula III include:

(a) compounds of the formula III wherein R¹, R², R³ and R⁴ are selected, independently, from (C₁-C₆)alkyl;

(b) compounds of the formula III wherein R³ and R⁴ are selected, independently, from (C₁-C₆)alkyl, and R¹ and R², together with the nitrogen to which they are attached, form a ring;

(c) compounds of the formula III wherein one of R¹ and R² is selected from (C₁-C₆)alkyl, and the other is selected from phenyl or phenyl-(C₁-C₆)alkyl;

(d) compounds of the formula III wherein R¹ and R², together with the nitrogen to which they are attached, form a piperazine, piperidine or pyrrolidine ring; and

(e) compounds of the formula III wherein R¹ and R² are selected, independently from (C₁-C₆)alkyl, and R³ and R⁴, together with the carbon to which they are attached, form a ring.

Examples of preferred compounds of the formula III are:

6-[2-Isopropoxy-4-((4-phenethylpiperazin-1-yl)-ethyl)-phenyl]-pyridin-2-ylamine;

6-[2-Isobutoxy-4-((4-phenethylpiperazin-1-yl)-ethyl)-phenyl]-pyridin-2-ylamine;

6-[2-Isobutoxy-4-((4-dimethylaminoethyl)-phenyl]-pyridin-2-ylamine;

6-[2-Isopropoxy-(N-(2-methyl)propyl)-4-(pyrrolidin-3-yl)-phenyl]-pyridin-2-ylamine;

1-[4-(6-Amino-pyridin-2-yl)-3-isopropoxy-phenyl]-2-(4-phenethyl-piperazin-1-yl)-ethanol;

6-[2-Cyclopentyloxy-4-((4-dimethylaminoethyl)-phenyl]-pyridin-2-ylamine;

6-[2-Cyclopentyloxy-4-((4-phenethylpiperazin-1-yl)-ethyl)-phenyl]-pyridin-2-ylamine;

and the pharamaceutically acceptable salts of the foregoing compounds.

Other examples of specific compounds of the formula III are:

6-[2-Cyclohexyloxy-4-((4-phenethylpiperazin-1-yl)-ethyl)-phenyl]-pyridin-2-ylamine;

6-[2-Cyclobutyloxy-4-((4-phenethylpiperazin-1-yl)-ethyl)-phenyl]-pyridin-2-ylamine;

6-[2-Cyclopropyloxy-4-((4-phenethylpiperazin-1-yl)-ethyl)-phenyl]-pyridin-2-ylamine;

6-[2-Isopentyloxy-4-((4-phenethylpiperazin-1-yl)-ethyl)-phenyl]-pyridin-2-ylamine;

6-[2-Isohexyloxy-4-((4-phenethyl piperazin-1-yl)-ethyl)-phenyl]-pyridin-2-ylamine;

6-[2-Cyclopentyloxy-(N-(2-methyl)propyl)-4-(pyrrolidin-3-yl)-phenyl]-pyridin-2-ylamine;

6-[2-Cyclohexyloxy-(N-(2-methyl)propyl)-4-(pyrrolidin-3-yl)-phenyl]-pyridin-2-ylamine;

6-[2-Cyclobutyloxy-(N-(2-methyl)propyl)-4-(pyrrolidin-3-yl)-phenyl]-pyridin-2-ylamine;

6-[2-Cyclopropyloxy-(N-(2-methyl)propyl)-4-(pyrrolidin-3-yl)-phenyl]-pyridin-2-ylamine;

6-[2-Isopentyloxy-(N-(2-methyl)propyl)-4-(pyrrolidin-3-yl)-phenyl]-pyridin-2-ylamine;

6-[2-Isohexyloxy-(N-(2-methyl)propyl)-4-(pyrrolidin-3-yl)-phenyl]-pyridin-2-ylamine;

1-[4-(6-Amino-pyridin-2-yl)-3-isobutoxy-phenyl]-2-(4-phenethyl-piperazin-1-yl)-ethanol;

1-[4-(6-Amino-pyridin-2-yl)-3-isopropoxy-phenyl]-2-(6,7-dimethoxy-tetrahydroisoquinol-2-yl)-ethanol;

1-[4-(6-Amino-pyridin-2-yl)-3-isopropoxy-phenyl]-2-(4-dimethylamino-piperidin-1-yl)-ethanol;

1-[4-(6-Amino-pyridin-2-yl)-3-isopropoxy-phenyl]-2-(dimethylamino)-ethanol; and

1-[4-(6-Amino-pyridin-2-yl)-3-cyclopenyloxy-phenyl]-2-(4-phenethyl-piperazin-1-yl)-ethanol;

and the pharmaceutically acceptable salts of the foregoing compounds.

Other examples of NOS inhibiting compounds that can be used in the methods and pharmaceutical compositions of this invention are compounds of the formula

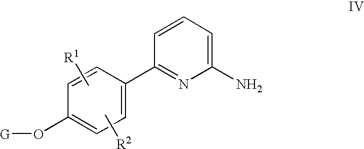

wherein R¹ and R² are selected, independently, from hydrogen, halo, hydroxy, (C₁-C₆)alkoxy, (C₁-C₇)alkyl, (C₂-C₆)alkenyl, and (C₂-C₁₀)alkoxyalkyl; and

G is selected from hydrogen, (C₁-C₆)alkyl, (C₁-C₆)alkoxy-(C₁-C₃)alkyl, aminocarbonyl-(C₁-C₃)alkyl-, (C₁-C₃) alkylaminocarbonyl-(C₁-C₃)alkyl-, di-[(C₁-C₃)alkyl]aminocarbonyl-(C₁-C₃)alkyl-, and N(R³)(R⁴)(C₀-C₄)alkyl-, wherein R³ and R⁴ are selected, independently, from hydrogen, (C₁-C₇)alkyl, tetrahydronaphthalene and aralkyl, wherein the aryl moiety of said aralkyl is phenyl or naphthyl and the alkyl moiety is straight or branched and contains from 1 to 6 carbon atoms, and wherein said (C₁-C₇) alkyl and said tetrahydronaphthalene and the aryl moiety of said aralkyl may optionally be substituted with from one to three substituents, preferably from zero to two substituents, that are selected, independently, from halo, nitro, hydroxy, cyano, amino, (C₁-C₄)alkoxy, and (C₁-C₄) alkylamino;

or R³ and R⁴ form, together with the nitrogen to which they are attached, a piperazine, piperidine, azetidine or pyrrolidine ring or a saturated or unsaturated azabicyclic ring system containing from 6 to 14 ring members, from 1 to 3 of which are nitrogen, from zero to two of which are oxygen, and the rest of which are carbon;

and wherein said piperazine, piperidine, azetidine and pyrrolidine rings and said azabicyclic ring systems may optionally be substituted with one or more substituents, preferably with from zero to two substituents, that are selected, independently, from (C₁-C₆)alkyl, amino, (C₁-C₆) alkylamino, [di-(C₁-C₆)alkyl]amino, phenyl substituted 5 to 6 membered heterocyclic rings containing from 1 to 4 ring nitrogen atoms, benzoyl, benzoylmethyl, benzylcarbonyl, phenylaminocarbonyl, phenylethyl and phenoxycarbonyl, and wherein the phenyl moieties of any of the foregoing substituents may optionally be substituted with one or more substituents, preferably with from zero to two substituents, that are selected, independently, from halo, (C₁-C₃)alkyl, (C₁-C₃)alkoxy, nitro, amino, cyano, CF₃ and OCF₃;

and wherein said piperazine, piperidine, azetidine and pyrrolidine rings and said azabicyclic ring systems may be attached to —(C₀-C₄)alkyl-O— (wherein the oxygen of said —(C₀-C₄)alkyl-O— is the oxygen atom depicted in structural formula II) at a nitrogen atom of the NR³R⁴ ring or at any other atom of such ring having an available bonding site;

or G is a group of the formula A

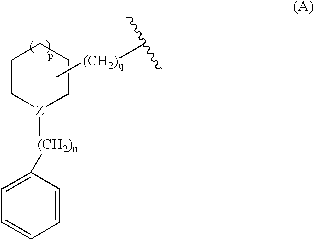

wherein Z is nitrogen or CH, n is zero or one, q is zero, one, two or three and p is zero, one or two;

and wherein the 2-amino piperidine ring depicted in structure I above may optionally be replaced with

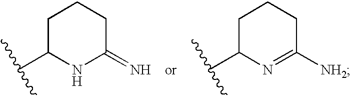

and the pharmaceutically acceptable salts of such compounds.

Examples of compounds of the formula IV are those wherein G is N(R³)(R⁴)(C₀-C₄) alkyl and N(R³)(R⁴) is amino, dimethylamino, methylbenzylamino, (C₁-C₄)alkylamino, di-[(C₁-C₄)alkyl]amino or one of the following groups:

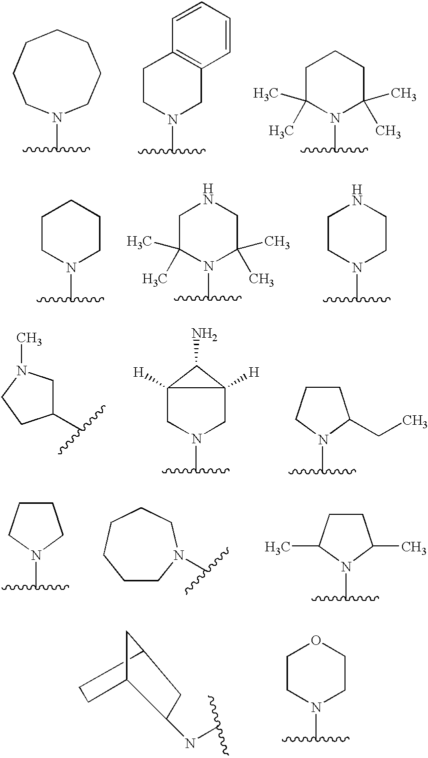

Preferred compounds of the formula IV include those wherein R² is hydrogen and R¹ is (C₁-C₃)alkoxy and is in the ortho position relative to the pyridine ring of formula IV.

Other compounds of the formula IV are those wherein G is a group of the formula A, as defined above, wherein Z is nitrogen.

Other compounds of the formula IV are those wherein R¹ and R² are selected, independently, from (C₁-C₂)alkoxy.

Other compounds of the formula IV are those wherein G is a group of the formula A, as defined above, wherein Z is nitrogen, each of p and n is one and q is two.

Other compounds of the formula IV are those wherein the 2-aminopyridine ring depicted in formula IV above is present.

Other examples of NOS inhibitors that can be used in the methods and pharmaceutical compositions of this invention are compounds of the formula

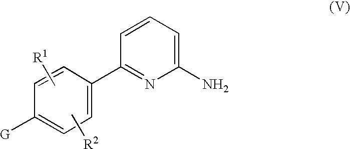

wherein R¹ and R² are selected, independently, from hydrogen, hydroxy, methyl and methoxy; and G is a group of the formula

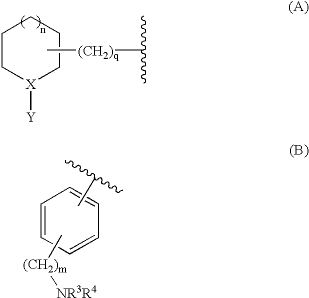

wherein

n is zero or one;

Y is NR³R⁴, (C₁-C₆)alkyl or aralkyl, wherein the aryl moiety of said aralkyl is phenyl or naphthyl and the alkyl moiety is straight or branched and contains from 1 to 6 carbon atoms, and wherein said (C₁-C₆))alkyl and the aryl moiety of said aralkyl may be substituted with from one to three substituents, preferably from zero to two substituents, that are selected, independently, from halo (e.g., chloro, fluoro, bromo or iodo), nitro, hydroxy, cyano, amino, (C₁-C₄)alkoxy and (C₁-C₄) alkylamino;

X is N when Y is (C₁-C₆)alkyl, aralkyl, or substituted (C₁-C₆)alkyl, and X is CH when Y is NR³R⁴;

q is zero, one or two;

m is zero, one or two; and

R³ and R⁴ are selected, independently, from (C₁-C₆) alkyl, tetrahydronaphthalene and aralkyl, wherein the aryl moiety of said aralkyl is phenyl or naphthyl and the alkyl moiety is straight or branched and contains from 1 to 6 carbon atoms, and wherein said (C₁-C₆) alkyl and said tetrahydronaphthalene and the aryl moiety of said aralkyl may optionally be substituted with from one to three substituents, preferably from zero to two substituents, that are selected, independently, from halo (e.g., chloro, fluoro, bromo or iodo), nitro, hydroxy, cyano, amino, (C₁-C₄) alkoxy, and (C₁-C₄) alkylamino;

or R³ and R⁴ form, together with the nitrogen to which they are attached, a piperazine, piperidine or pyrrolidine ring or an azabicyclic ring containing from 6 to 14 ring members, from 1 to 3 of which are nitrogen and the rest of which are carbon, wherein an example of said azabicyclic rings is the 3-aza-bicyclo[3.1.0]hex-6-ylamine ring;

and wherein said piperazine, piperidine and pyrrolidine rings may optionally be substituted with one or more substituents, preferably with from zero to two substituents, that are selected, independently, from amino, (C₁-C₆) alkylamino, [di-(C₁-C₆)alkyl]amino, phenyl substituted 5 to 6 membered heterocyclic rings containing from 1 to 4 rings nitrogen atoms, benzoyl, benzoylmethyl, benzylcarbonyl, phenylaminocarbonyl, phenylethyl and phenoxycarbonyl, and wherein the phenyl moieties of any of the foregoing substituents may optionally be substituted with one or more substituents, preferably with from zero to two substituents, that are selected, independently, from halo, (C₁-C₃)alkyl, (C₁-C₃)alkoxy, nitro, amino, cyano, CF₃ and OCF₃;

and the pharmaceutically acceptable salts of such compounds.

Examples of preferred compounds of the formula V are those wherein NR³R⁴ is:

4-phenylethylpiperazin-1-yl;

4-methylpiperazin-1-yl;

phenethylamino; or

3-aza-bicyclo[3.1.0]hex-6-ylamine.

Other preferred compounds of the formula V are those wherein NR³R⁴ is a group of the formula

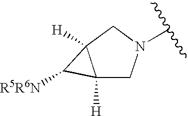

wherein NR⁵R⁶ is NH₂.

Other examples of NOS inhibitors that can be employed in the methods and pharmaceutical composition of this invention are compounds of the formula

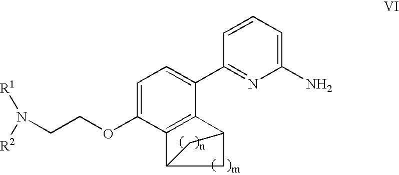

wherein n and m in the bridging rings are independently 1, 2 or 3, and a carbon in one of said bridging rings may be substituted by a heteroatom selected from O, S and N, with the proviso that a bridgehead carbon can only be substituted by nitrogen, and R¹ and R² are independently selected from C₁ to C₆ alkyl, which may be linear, branched or cyclic or contain both linear and cyclic or branched and cyclic moieties, wherein each of R¹ and R² may be independently optionally substituted with from one to three substituents, preferably from zero to two substituents, that are selected, independently, from halo (e.g., chloro, fluoro, bromo, iodo), nitro, hydroxy, cyano, amino, (C₁-C₄) alkoxy, and (C₁-C₄) alkylamino;

or R¹ and R² form, together with the nitrogen to which they are attached, a piperazine, azetidine, piperidine or pyrrolidine ring or an azabicyclic ring containing from 6 to 14 ring members, from 1 to 3 of which are nitrogen and the rest of which are carbon,

wherein the distal nitrogen on said piperazine or azabicylic ring is optionally substituted with groups R³ and R⁴ wherein R³ and R⁴ are selected from hydrogen, C₁ to C₆ alkyl, phenyl, naphthyl, C₁ to C₆ alkyl-C(═O)—, HC(═O)—, C₁ to C₆ alkoxy-(C═O)—, phenyl-C(═O)—, naphthyl-C(═O)—, and R⁶R⁷NC(═O)— wherein R⁶ and R⁷ are selected, independently, from hydrogen and C₁ to C₆ alkyl, with the proviso that when said azabicyclic ring is a spirocyclic ring, the distal nitrogen on said spirocyclic ring is optionally substituted with R⁵ wherein R⁵ is selected from hydrogen, C₁ to C₆ alkyl, phenyl, naphthyl, phenyl-C₁ to C₆ alkyl- and naphthyl C₁ to C₆ alkyl-;

and wherein said piperazine, azetidine, piperidine and pyrrolidine rings may optionally be substituted with one or more substituents, preferably with from zero to two substituents that are selected, independently, from C₁ to C₆ alkyl, amino, C₁ to C₆ alkylamino, [di-C₁-C₆ alkyl]amino, phenyl substituted 5 to 6 membered heterocyclic rings containing from 1 to 4 rings nitrogen atoms, benzoyl, benzoylmethyl, benzylcarbonyl, phenylaminocarbonyl, phenylethyl and phenoxycarbonyl, and wherein the phenyl moieties of any of the foregoing substituents may optionally be substituted with one or more substituents, preferably with from zero to two substituents, that are selected, independently, from halo, C₁ to C₃ alkyl, C₁ to C₃ alkoxy, nitro, amino, cyano, CF₃ and OCF₃;

with the proviso that no carbon atom is substituted with more than one substituent selected from hydroxy, amino, alkoxy, alkylamino and dialkylamino;

and the pharmaceutically acceptable salts of said compounds.

Examples of the azabicyclic rings that may be formed by NR¹R² in the above compounds of formula VI are

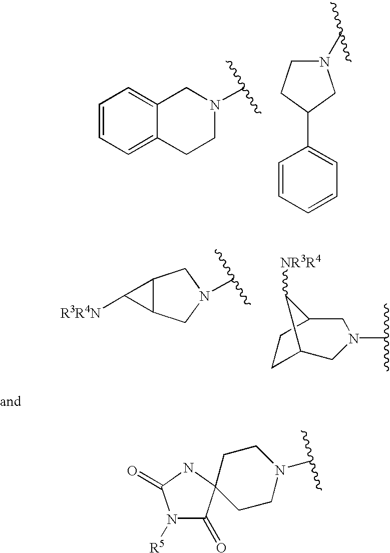

wherein R³ and R⁴ are selected from hydrogen, C₁ to C₆ alkyl, phenyl, naphthyl, C₁ to C₆ alkyl-C(═O)—, HC(═O)—, C₁ to C₆ alkoxy-(C═O)—, phenyl-C(═O)—, naphthyl-C(═O)—, and R⁶R⁷NC(═O)— wherein R⁶ and R⁷ are selected, independently, from hydrogen and C₁ to C₆ alkyl; and

R⁵ is selected from hydrogen, C₁ to C₆ alkyl, phenyl, naphthyl, phenyl-C₁ to C₆ alkyl- and naphthyl C₁ to C₆ alkyl-.

Preferred compounds of the formula IV include those wherein NR¹R² is an optionally substituted piperidine, azetidine, piperazine or pyrrolidine ring or a 3-aza-bicyclo[3.1.0]hex-6-ylamine ring;

and wherein said piperazine, azetidine, piperidine, pyrrolidine and 3-aza-bicyclo[3.1.0]hex-6-ylamine rings may optionally be substituted with one or more substituents, preferably with from zero to two substituents that are selected, independently, from C₁ to C₆ alkyl, amino, C₁ to C₆ alkylamino, [di-C₁ to C₆ alkyl]amino, phenyl, substituted 5 to 6 membered heterocyclic rings containing from 1 to 4 rings nitrogen atoms, benzoyl, benzoylmethyl, benzylcarbonyl, phenylaminocarbonyl, phenylethyl and phenoxycarbonyl, and wherein the phenyl moieties of any of the foregoing substituents may optionally be substituted with one or more substituents, preferably with from zero to two substituents, that are selected, independently, from halo, C₁ to C₃ alkyl, C₁ to C₃ alkoxy, nitro, amino, cyano, CF₃ and OCF₃;

and the pharmaceutically acceptable salts of said compounds.

The following compounds are preferred compounds of the formula VI:

6-[8-(2-Dimethylamino-ethoxy)-1,2,3,4-tetrahydro-1,4-methano-naphthalen-5-yl]-pyridin-2-ylamine; and

6-[8-(2-Pyrrolidin-1-yl-ethoxy)-1,2,3,4-tetrahydro-1,4-methano-naphthalen-5-yl]-pyridin-2-ylamine.

Other compounds of the formula VI are the following:

6-[8-(2-Dimethylamino-ethoxy)-1,2,3,4-tetrahydro-1,4-ethano-naphthalen-5-yl]-pyridin-2-ylamine;

6-[8-(2-Pyrrolidin-1-yl-ethoxy)-1,2,3,4-tetrahydro-1,4-ethano-naphthalen-5-yl]-pyridin-2-ylamine;

6-[8-(2-(4-Dimethylamino-piperidin-1-yl)-ethoxy)-1,2,3,4-tetrahydro-1,4-methano-naphthalen-5-yl]-pyridin-2-ylamine;

6-[8-(2-(6,7-Dimethoxy-teyrahydroisoquinol-2-yl)-ethoxy)-1,2,3,4-tetrahydro-1,4-methano-naphthalen-5-yl]-pyridin-2-ylamine; and

6-[8-(2-(4-Methylpiperazin-1-yl)-ethoxy)-1,2,3,4-tetrahydro-1,4-methano-naphthalen-5-yl]-pyridin-2-ylamine.

Compounds of formulas I-VI may contain chiral centers and therefore may exist in different enantiomeric and diastereomeric forms. This invention relates to the above methods of treatment using and the above pharmaceutical compositions comprising all optical isomers and all stereoisomers of compounds of the formulas I-V and mixtures thereof.

The term “alkyl”, as used herein, unless otherwise indicated, includes saturated monovalent hydrocarbon radicals having straight, branched or cyclic moieties or combinations thereof.

The term “one or more substituents”, as used herein, refers to a number of substituents that equals from one to the maximum number of substituents possible based on the number of available bonding sites.

The terms “halo” and “halogen”, as used herein, unless otherwise indicated, include chloro, fluoro, bromo and iodo.

Formulas I-VI above include compounds identical to those depicted but for the fact that one or more hydrogen, carbon or other atoms are replaced by isotopes thereof. Such compounds may be useful as research and diagnostic tools in metabolism pharmacokinetic studies and in binding assays.

### DETAILED DESCRIPTION OF THE INVENTION

In the discussion that follows, formulas I, II, III, IV, V and VI are defined as set forth above in the Summary of the Invention.

Compounds of the formula I and their pharmaceutically acceptable salts can be prepared as described below and in U.S. provisional application No. 60/057094, which was filed Aug. 27, 1997 and is entitled “2-Aminopyrindines Containing Fused Ring Substituents”, and in the PCT application having the same title that was filed on May 5, 1998, which designates the United States and claims priority from provisional application No. 60/057094.

In Schemes 1-3 and the discussion of Schemes 1-3 that follow, all substituents are defined as they are defined above for compounds of the formula I.

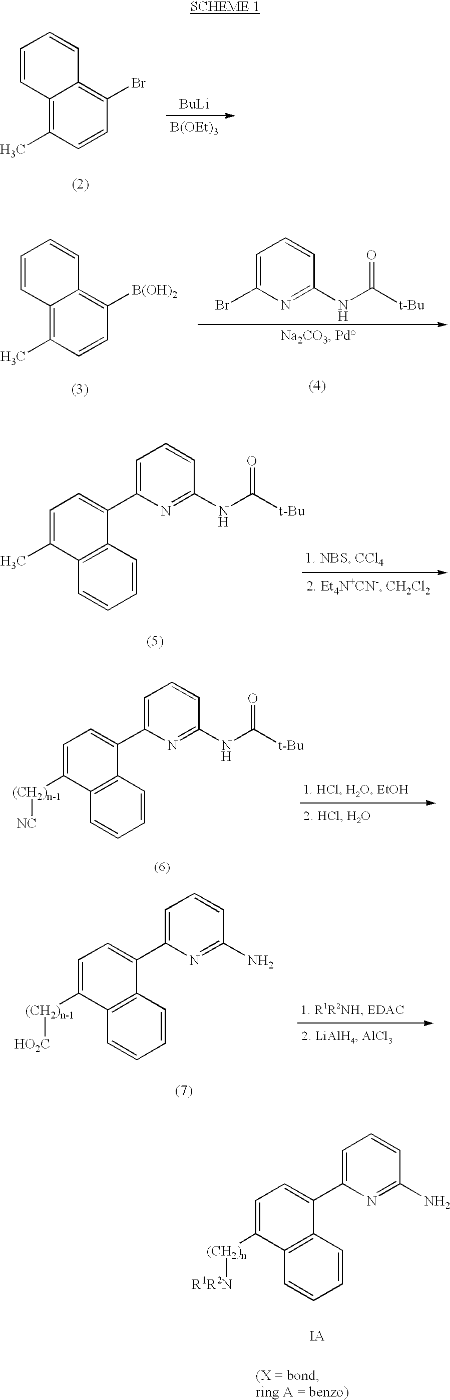

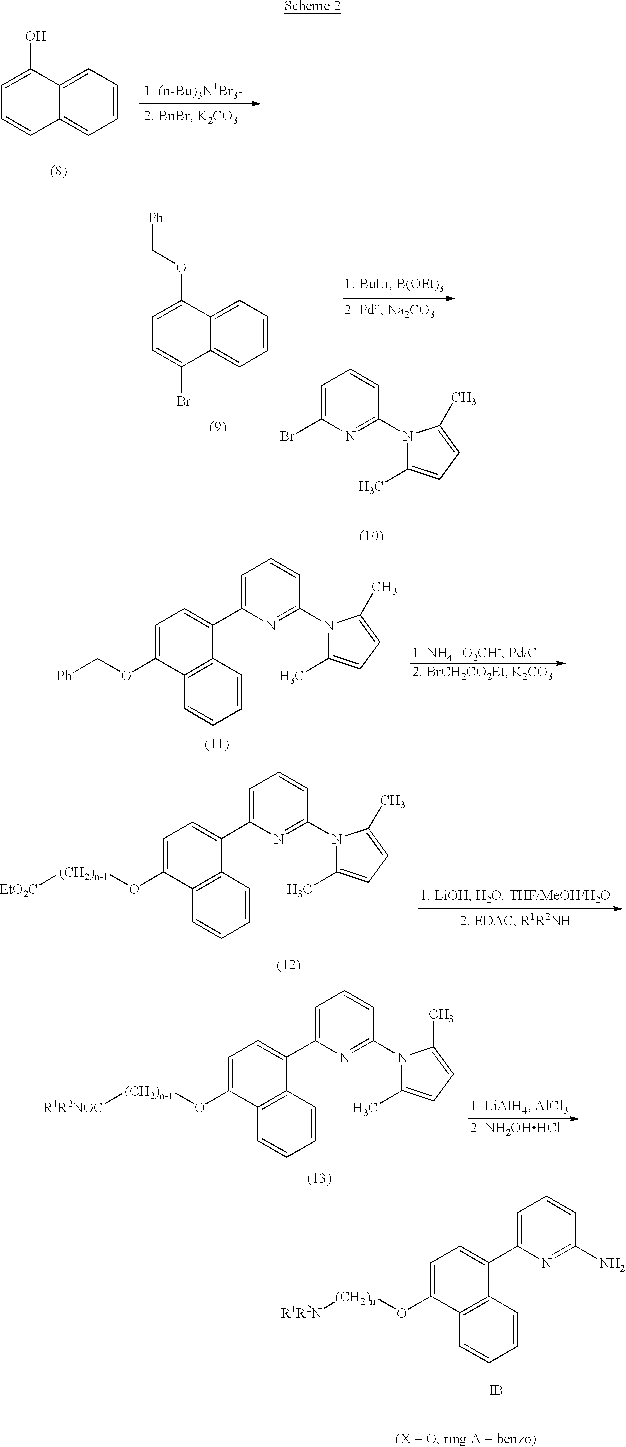

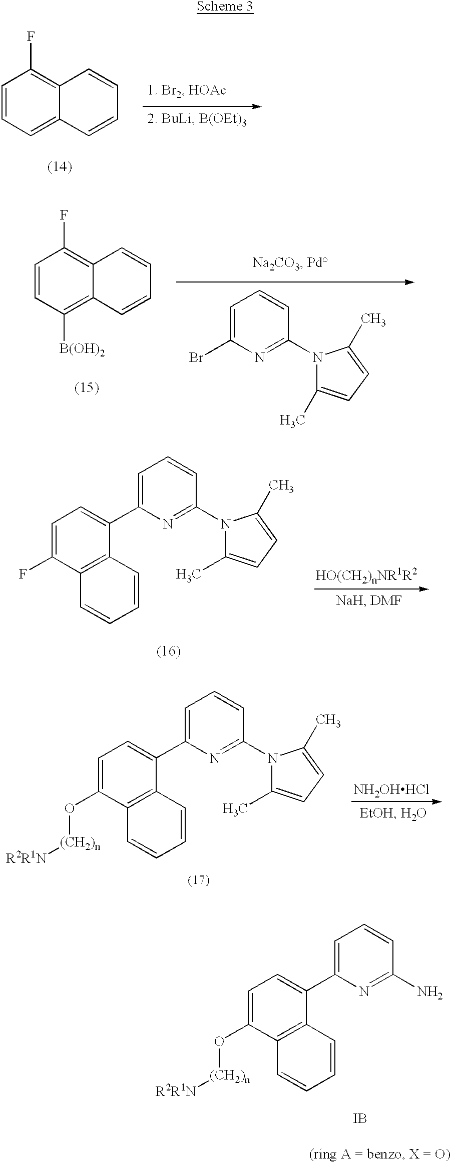

Scheme 1 illustrates a method of preparing compounds of the formula I wherein X is a bond and ring A is benzo. Schemes 2 and 3 illustrate methods of preparing compounds of the formula I wherein X is oxygen and ring A is benzo. The starting materials used in the procedures of Schemes 1 and 2 are either commercially available, known in the art or readily obtainable from known compounds by methods that will be apparent to those skilled in the art.

Referring to Scheme 1, the compound of formula (2) is cooled to about 70° C. in dry tetrahydrofuran (THF), and then a solution of n-butyl lithium is added to it. The resulting solution is then treated with triethyl borate and allowed to warm to room temperature to form the compound of formula (3).

The compound of formula (3) is reacted with the compound of formula (4) to form the compound of formula (5). This reaction is generally carried out in an aqueous ethanol solvent, in the presence of sodium carbonate and tetrakistriphenylphoshine palladium, at about the reflux temperature.

The compound of the formula (6) can be formed in the following manner. First, the compound of formula (5) is reacted with N-bromosuccinimide (NBS) and bis-(1-cyano-1-aza)-cyclohexane in carbon tetrachloride and refluxed for about 8 hours, with additional portions of the initiator being added at about 1, 2 and 4 hours. After evaporation of the solvent, the product of this reaction is reacted with triethylammonium cyanide in methylene chloride at about room temperature to form the compound of formula (6).

Saturation of a solution of the compound of formula (6) in ethanol with hydrogen chloride, followed by refluxing the mixture and then heating in aqueous hydrochloric acid, yields the compound of formula (7).

The compound of the formula (7) that is formed in the preceding step can be converted into the compound of formula IA in the following manner. First, the compound of formula (7) is reacted with the appropriate compound of the formula R²R¹NH and N-ethyl-N-dimethylaminopropyl carbodiimide (EDAC) in the presence of a base. Examples of suitable bases are those selected from trialkylamines, alkali metal carbonates and alkaline earth metal carbonates. This reaction is typically conducted in a solvent such as acetonitrile, methylene chloride or N,N-dimethylformamide (DMF), at a temperature from about room temperature to about 100° C., preferably at about room temperature. Preferably, the reaction is conducted in the presence of a catalytic additive such as N-hydroxysuccinamide or hydroxybenzotriazole.

The product of the foregoing reaction is then reduced using methods well known to those of skill in the art. For example, the reduction can be carried out using lithium aluminum hydride in tetrahydrofuran, with or without aluminum chloride, or using borane methyl sulfide in tetrahydrofuran, at a temperature of about −78° C. to about 0° C., preferably at about −70° C., to yield the desired compound of formula IA.

Referring to scheme 2, the compound of formula (8) is reacted with tetrabutylammonium tribromide in 1,2-dichloroethane at about room temperature. The product of this reaction is then treated with benzyl bromide and potassium carbonate in a solvent such as acetonitite, at about the reflux temperature of the reaction mixture, to form the compound of formula (9).

The compound of formula (9) is then converted into 1-benzyloxy-naphthalene-4-boronic acid by the procedure described above for preparing the boronic acid derivative of formula (3) in Scheme 1.

Reaction of 1-benzyoxy-napthalene-4-boronic acid with the compound of formula (10) in an ethanol solvent, in the presence of sodium carbonate and tetrakistriphenyl palladium, at about the reflux temperature of the reaction mixture, yields the compound of formula (11).

The compound of formula (11) can be converted into the compound of formula (13) using the following two step process. The compound of formula (11) is reacted with ammonium formate and ten percent palladium on carbon, in an ethanol solvent, at about the reflux temperature of the reaction mixture, to yield the analogous compound to that having formula (11), wherein the benzyloxy group of formula (11) is replaced with a hydroxy group. The compound of formula (12) is then formed by reacting the above hydroxy derivative with 2-bromoethylacetate and potassium carbonate in acetonitrile at about the reflux temperature of the reaction mixture.

Basic hydrolysis of the compound of formula (12), followed by reaction with N-ethyl-N-3-dimethylaminopropylcarbodiimide (EDAC) and the appropriate compound having the formula R¹R²NH yields the desired compound of the formula (13). The base hydrolysis is typically carried out using an alkali metal or alkaline earth metal hydroxide in a mixture of THF, methanol and water at about room temperature. The reaction with R¹R²NH and EDAC is generally carried out using the procedure described above for the preparation of compounds of the formula IA from those of formula (7) in Scheme 1.

The compound of formula (13) can be converted into the desired compound of formula IB as follows. The compound of formula (13) is reduced to form the corresponding compound wherein the carbonyl group is replaced by a methylene group, after which the 2,5-dimethylpyrrolyl protecting group is removed. The reduction can be carried out using methods well known to those of skill in the art, for example, using lithium aluminum hydride in tetrahydrofuran, with or without aluminum chloride, or using borane methyl sulfide in tetrahydrofuran, at a temperature of about −78° C. to about 0° C., preferably at about −70° C.

Removal of the 2,5-dimethylpyrrolyl protecting group can be accomplished by reaction with hydroxylamine hydrochloride. This reaction is generally carried out in an alcoholic or aqueous alcoholic solvent, at a temperature from about room temperature to about the reflux temperature of the reaction mixture, preferably at about the reflux temperature, for about 8 to about 72 hours.

Compounds of the formula I that are identical to those of formula IB but for the fact that ring A is other than benzo can be prepared in an analogous fashion, starting with the appropriate compound that is analogous to that of formula (8), wherein the unsubstituted benzo ring of formula (8) is replaced by a ring other than benzo that is within the definition of ring A.

Referring to Scheme 3, the known 1-fluoronaphthalene (14) is brominated with bromine in acetic acid at a temperature from about room temperature to about the reflux temperature of the reaction mixture for about 1 to about 48 hours, and the bromide cooled to about −70° C. in dry tetrahydrofuran (THF), and then a solution of n-butyl lithium is added to it. The resulting solution is then treated with triethyl borate and allowed to warm to room temperature to form the compound of formula (15). The compound of formula (15) is reacted with the compound of formula (4) to form the compound of formula (16). This reaction is generally carried out in an aqueous ethanol solvent, in the presence of sodium carbonate and tetrakistriphenylphoshine palladium, at about the reflux temperature. The compound of formula (16) is then treated with an alkali metal alkoxide prepared from a compound of the formula HO(CH₂)ₙNR¹R² and sodium hydride in a polar solvent such as dimethylformamide, at a temperature from room temperature to 140° C. for about 1 to about 48 hours. This reaction produces the corresponding compound of formula (17), which is then deblocked to remove the 2,5-dimethylpyrrolyl protecting group by reaction with hydroxylamine hydrochloride. This reaction is generally carried out in an alcoholic or aqueous alcoholic solvent, at a temperature from about room temperature to about the reflux temperature of the reaction mixture, preferably at about the reflux temperature, for about 8 to about 72 hours.

Compounds of the formula I that are identical to those of formula IA and IB but for the fact that ring A is other than benzo can be prepared in an analogous fashion, starting with the appropriate starting materials that are analogous to those of formulas (2), (8) and (14), in Schemes 1, 2 and 3, respectively, wherein the unsubstituted benzo ring of such starting materials is replaced by a ring other than benzo that is within the definition of ring A.

The preparation of other compounds of the formula I not specifically described in the foregoing experimental section can be accomplished using combinations of the reactions described above that will be apparent to those skilled in the art.

In each of the reactions discussed or illustrated above, pressure is not critical unless otherwise indicated. Pressures from about 0.5 atmospheres to about 5 atmospheres are generally acceptable, and ambient pressure, i.e., about 1 atmosphere, is preferred as a matter of convenience.

Compounds of the formula II and their pharmaceutically acceptable salts can be prepared as described in published PCT patent application WO 97/36871, which designates the United States and was published on Oct. 9, 1997. The foregoing application is incorporated herein by reference in its entirety.

Compounds of the formula III and their pharmaceutically acceptable salts can be prepared as described below and in U.S. provisional patent application No. 60/057739 of John A. Lowe, III, entitled “6-Phenylpyridin-2-yl-amine Derivatives”, which was filed on Aug. 28, 1997. The foregoing application is incorporated herein by reference in its entirety.

Schemes 4 and 5 below illustrate methods of preparing compounds of the formula III.

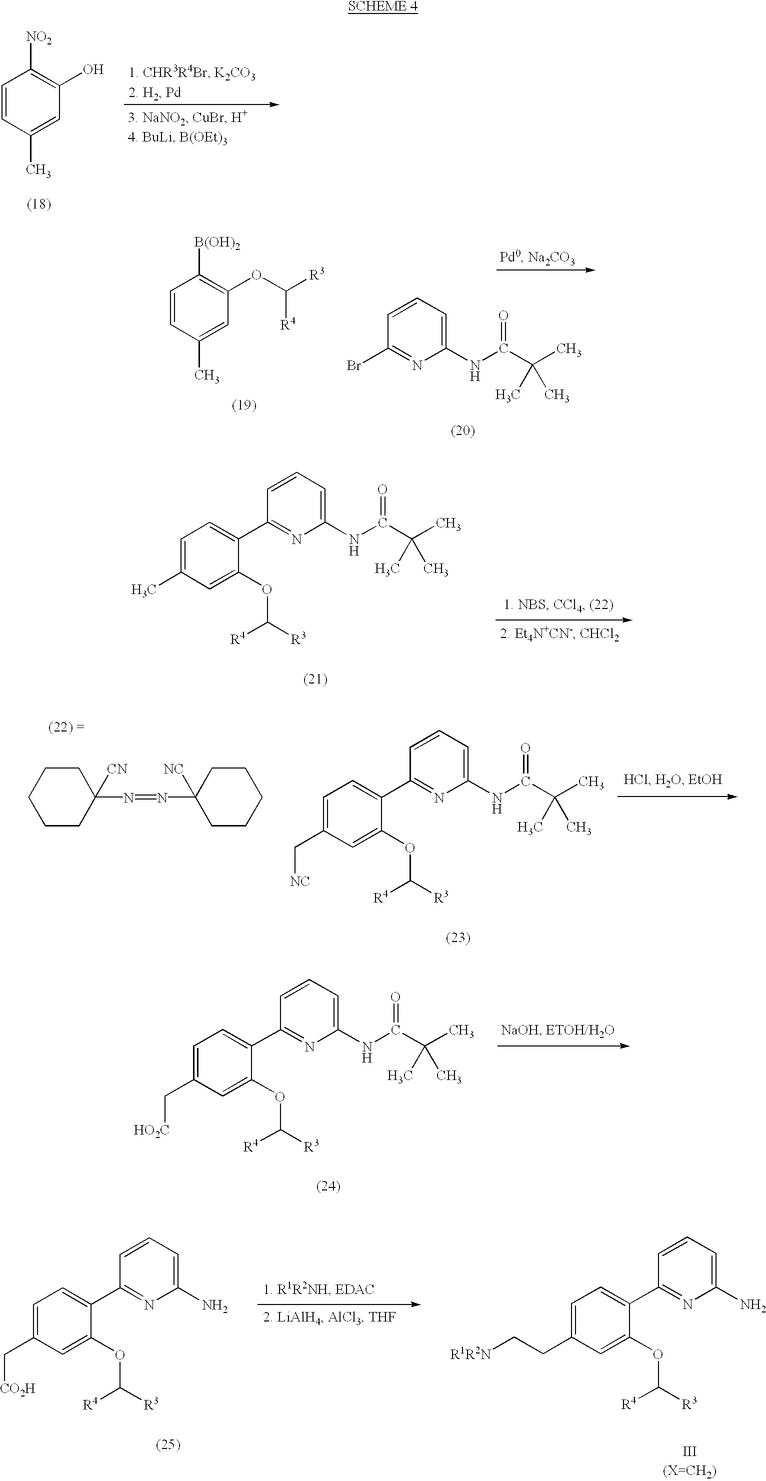

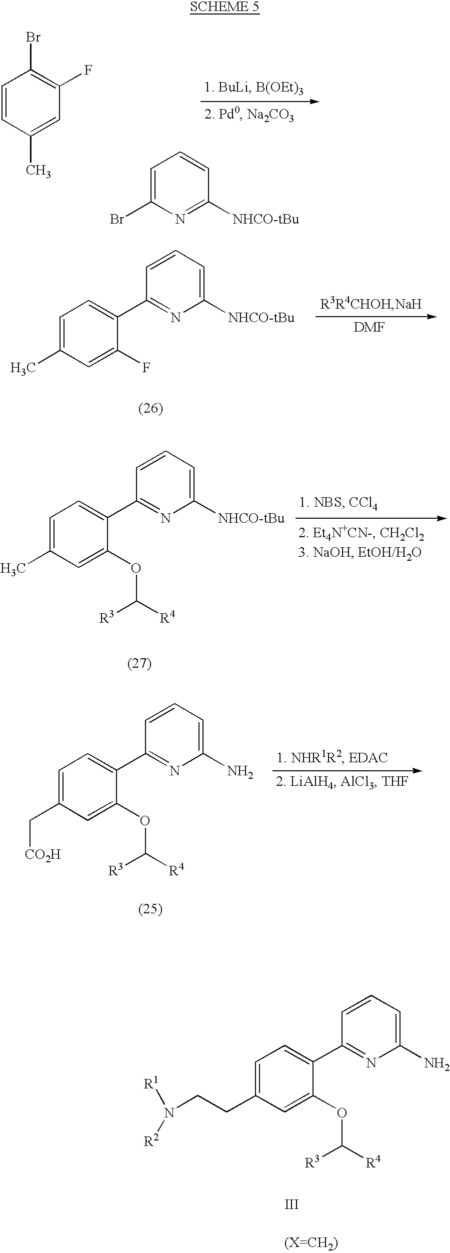

Referring to Scheme 4, the compound of formula (18) is reacted with a compound of the formula CHR³R⁴Br or CHR²R⁴I and potassium carbonate, in a solvent such as acetonitrile, at about the reflux temperature of the reaction mixture, to convert the hydroxy group of formula (18) into a group having the formula —OCHR³R⁴. The resulting compound is then reduced, at about room temperature, using hydrogen gas in the presence of 10% palladium on carbon, in an ethanol solvent, to form 3-OCHR³R⁴-4-aminotoluene, which is then reacted with sodium nitrite and cuprous bromide in concentrated sulfuric acid to form 3-OCHR³R⁴-4-bromotoluene.

The 3-OCHR³R⁴-4-bromotoluene produced in the foregoing reaction is then cooled to about −70° C. in dry tetrahydrofuran (THF), and a solution of n-butyl lithium is added to it. The resulting solution is then treated with triethyl borate and allowed to warm to room temperature to form the compound of formula (19).

The compound of formula (19) is reacted with the compound of formula (20) to form the compound of formula (21). This reaction is generally carried out in an aqueous ethanol solvent, in the presence of sodium carbonate and tetrakistriphenylphosphine palladium, at about the reflux temperature of the reaction mixture.

The compound of the formula (23) can be formed in the following manner. First, the compound of formula (21) is reacted with N-bromosuccinimide (NBS) and bis-(1-cyano-1-aza)-cyclohexane (formula (22)) in carbon tetrachloride and refluxed for about 8 hours, with additional portions of the initiator being added at about 1, 2 and 4 hours. After evaporation of the solvent, the product of this reaction is reacted with triethylammonium cyanide in methylene chloride at about room temperature to form the compound of formula (23).

Saturation of a solution of the compound of formula (23) in ethanol with hydrogen chloride, followed by refluxing the mixture and then heating in aqueous hydrochloric acid, yields the compound of formula (24). Hydrolysis of the compound of formula VIII yields the corresponding compound of formula (25). The base hydrolysis is typically carried out using an alkali metal or alkaline earth metal hydroxide in a mixture of ethanol and water at a temperature from about room temperature to about the reflux temperature of the solvent.

The compound of the formula (25) that is formed in the preceding step can be converted into the compound of formula III (wherein X is CH₂) in the following manner. First, the compound of formula (25) is reacted with the appropriate compound of the formula R²R¹NH and N-ethyl-N-dimethylaminopropyl carbodiimide (EDAC) in the presence of a base. Examples of suitable bases are those selected from trialkylamines, alkali metal carbonates and alkaline earth metal carbonates. This reaction is typically conducted in a solvent such as acetonitrile, methylene chloride or N,N-dimethylformamide (DMF), at a temperature from about room temperature to about 100° C., preferably at about room temperature. Preferably, the reaction is conducted in the presence of a catalytic additive such as N-hydroxysuccinamide or hydroxybenzotriazole.

The product of the foregoing reaction is then reduced using methods well known to those of skill in the art. For example, the reduction can be carried out using lithium aluminum hydride in tetrahydrofuran, with or without aluminum chloride, or using borane methyl sulfide in tetrahydrofuran, at a temperature of about −78° C. to about 0° C., preferably at about −70° C., to yield the desired compound of formula III (wherein X is CH₂).

Referring to Scheme 5, 4-bromo-3-fluorotoluene is first converted to the boronic acid derivative and then coupled to 6-bromo-2-(t-butylcarbonylamino)pyridine to form compound of the formula (26) in the following manner. A halogen-metal exchange reaction is carried out on 3-fluoro-4-bromotoluene in tetrahydrofuran, ether, dimethoxyethane, hexane or another suitable ethereal or hydrocarbon solvent, at a temperature from −100° C. to about room temperature, using butyl lithium or another suitable alkyl lithium reagent, followed by reaction with a borate triester such as triethyl or triisopropyl borate, for about 1 to about 48 hours at a temperature from about −100° C. to about the reflux temperature. The intermediate boronic acid derivative is then converted into the compound of formula (26) in an aqueous ethanol solvent, in the presence of sodium carbonate and tetrakistriphenylphosphine palladium, at about the reflux temperature of the reaction mixture, using 6-bromo-2-(t-buylcarbonylamino)pyridine as the coupling partner. The compound of formula (26) is then converted into a compound of the formula (27) by displacement of the fluoro group from the alcohol with a suitable alkoxide, which is formed in a solvent such as dimethylformamide, tetrahydrofuran or dioxane, and a metal hydride such as sodium hydride, at a temperature from about room temperature to about the reflux temperature, for a period of about 5 minutes to about 5 hours. The reaction with the compound of formula (26) is carried out in this reaction system at a temperature from room temperature to about the reflux temperature for a period from about 1 to about 48 hours.

The compound of formula (27) is then converted into the corresponding compound of the formula (25) in the following manner. First, the compound of formula (27) is reacted with N-bromosuccinimide (NBS) and bis-(1-cyano-1-aza)-cyclohexane (formula (22) in Scheme 4) in carbon tetrachloride and refluxed for about 8 hours, with additional portions of the initiator being added after about 1, 2 and 4 hours, to brominate the methyl group of such compound. After evaporation of the solvent, the product of this reaction is reacted with triethylammonium cyanide in methylene chloride at about room temperature to form the corresponding compound wherein the bromo substituent is replaced by cyano. The resulting cyano derivative is then hydrolyzed to form the corresponding compound of formula (25). The base hydrolysis is typically carried out using an alkali metal or alkaline earth metal hydroxide in a mixture of ethanol and water at a temperature from about room temperature to about the reflux temperature of the solvent.

The compound of the formula (25) that is formed in the preceding step can be converted into the compound of formula I in the following manner. First, the compound of formula (25) is reacted with the appropriate compound of the formula R²R¹NH and N-ethyl-N-dimethylaminopropyl carbodiimide (EDAC) in the presence of a base. Examples of suitable bases are those selected from trialkylamines, alkali metal carbonates and alkaline earth metal carbonates. This reaction is typically conducted in a solvent such as acetonitrile, methylene chloride or N,N-dimethylformamide (DMF), at a temperature from about room temperature to about 100° C., preferably at about room temperature. Preferably, the reaction is conducted in the presence of a catalytic additive such as N-hydroxysuccinamide or hydroxybenzotriazole.

The product of the foregoing reaction is then reduced using methods well known to those of skill in the art to yield the desired compound of formula III (wherein X is CH₂). For example, the reduction can be carried out using lithium aluminum hydride in tetrahydrofuran, with or without aluminum chloride, or using borane methyl sulfide in tetrahydrofuran, at a temperature of about −78° C. to about 0° C., preferably at about −70° C.

Compounds of the formula III wherein X is CHOH can be prepared using a procedure analogous to that described in Example 1 of this application. Compounds of the formula I wherein X is part of a five or six membered saturated ring may be prepared using a procedure analogous to that described in Example 2.

The starting materials used in the procedures of Schemes 4 and 5 are either commercially available, known in the art or readily obtainable form known compounds by methods that will be apparent to those skilled in the art.

The preparation of other compounds of the formula III not specifically described in the foregoing experimental section can be accomplished using combinations of the reactions described above that will be apparent to those skilled in the art.

In each of the reactions discussed or illustrated above, pressure is not critical unless otherwise indicated. Pressures from about 0.5 atmospheres to about 5 atmospheres are generally acceptable, and ambient pressure, i.e., about 1 atmosphere, is preferred as a matter of convenience.

Compounds of the formula IV and their pharmaceutically acceptable salts can be prepared as described in PCT patent application PCT/IB98/00112, entitled “4-Amino-6-(2-substituted-4-phenoxy)-substituted-pyridines”, which designates the United States and was filed on Jan. 29, 1998. The foregoing application is incorporated herein by reference in its entirety.

Schemes 6-14 below illustrate methods of preparing compounds of the formula IV.

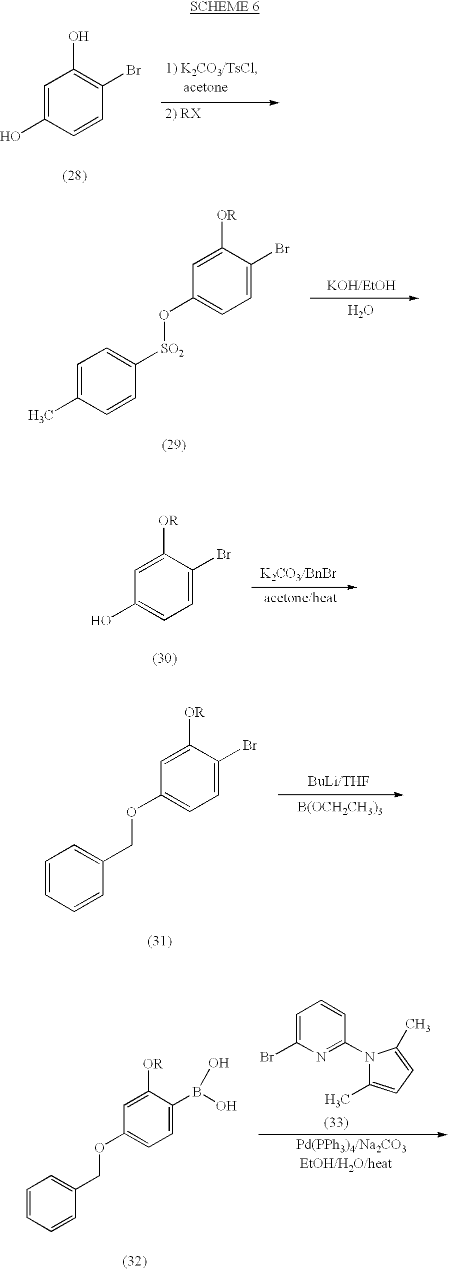

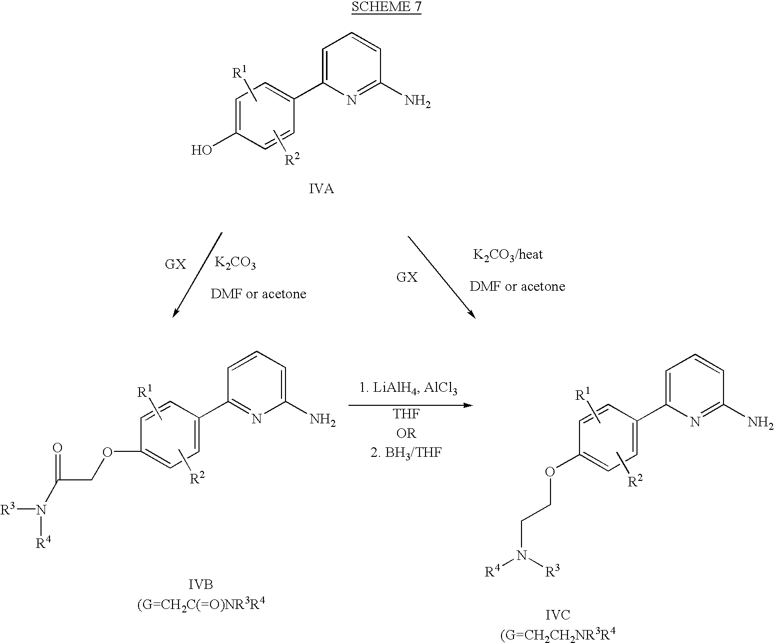

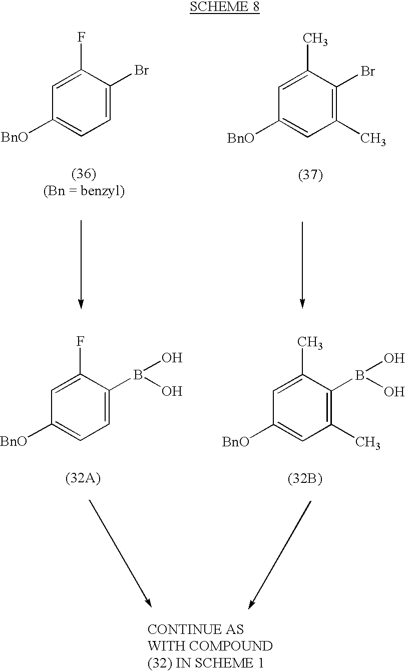

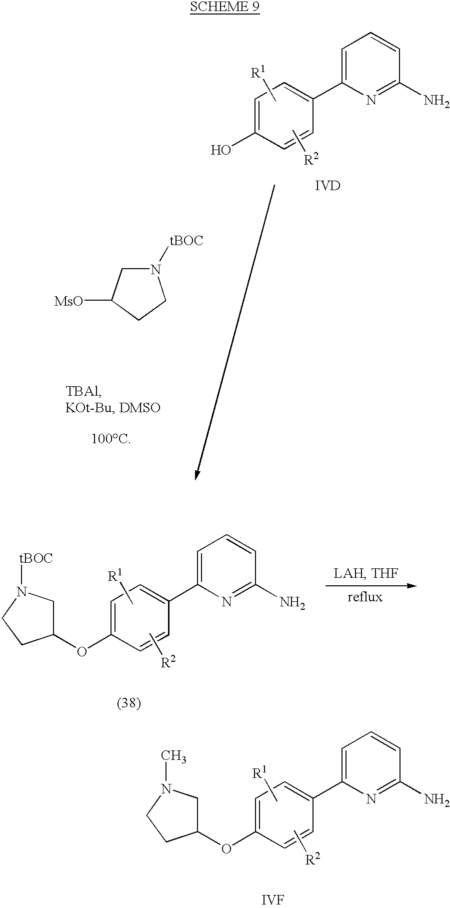

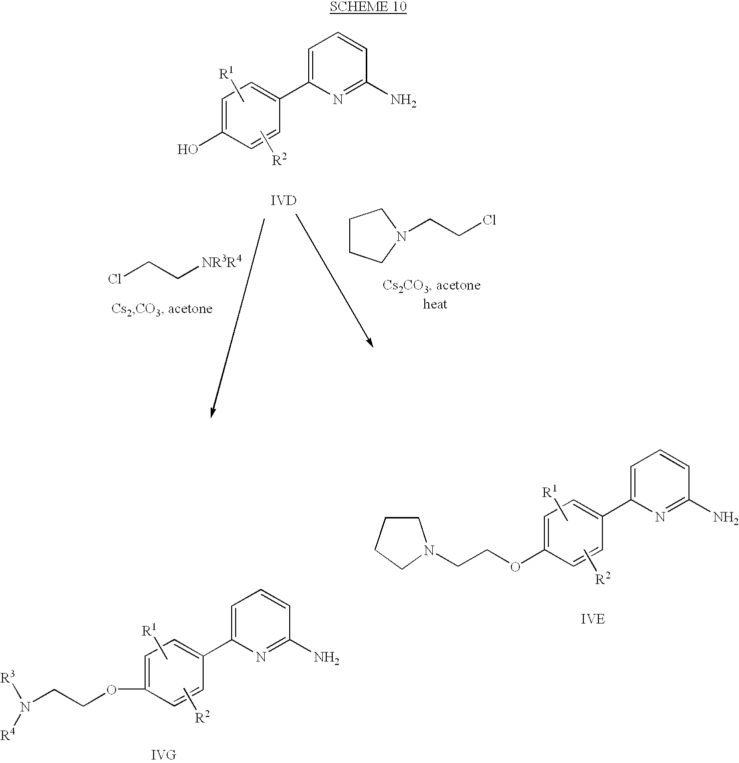

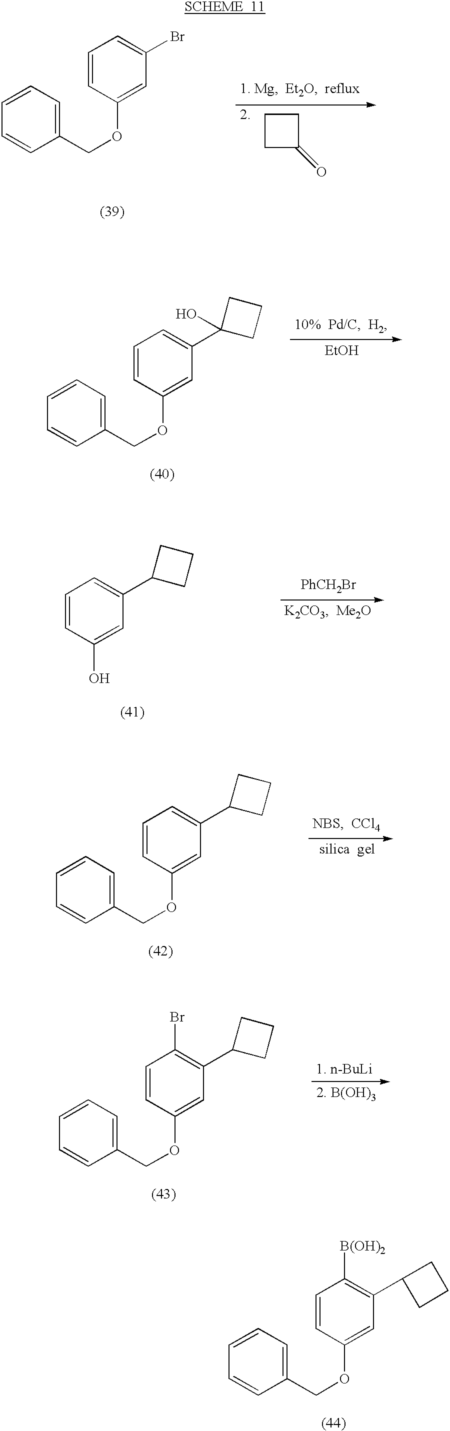

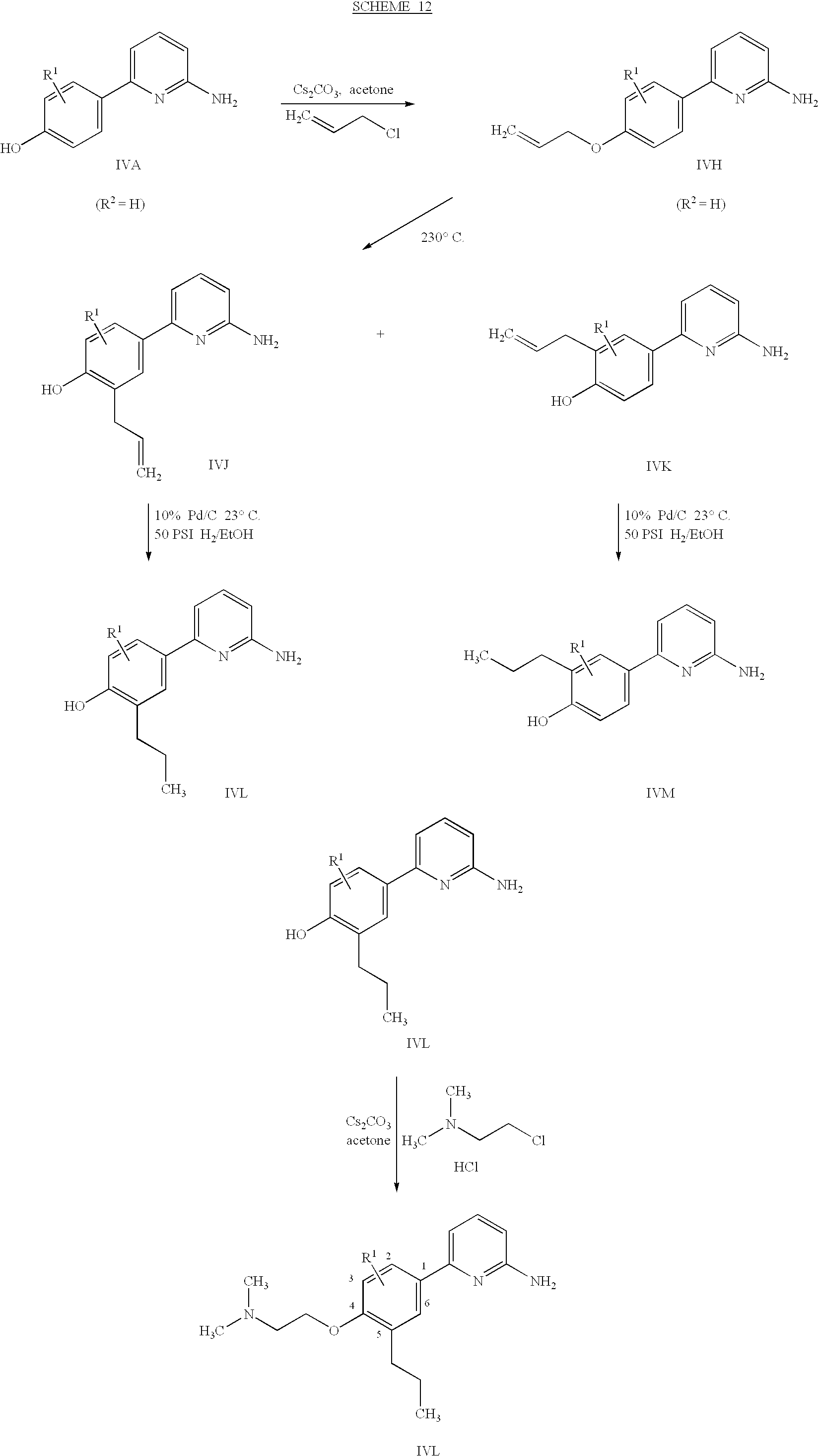

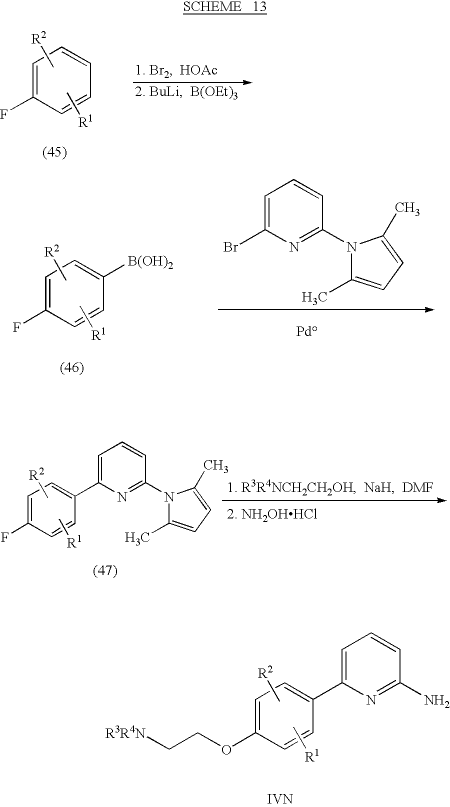

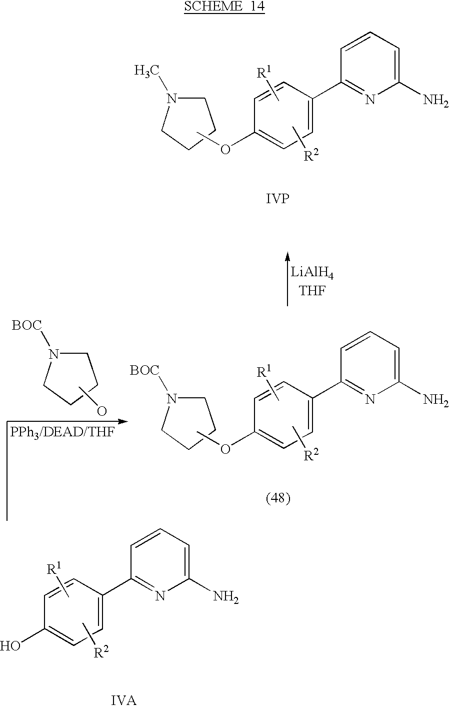

Scheme 6 illustrates a method for preparing compounds of the formula I wherein G is hydrogen, R¹ is —OR wherein R is (C₁-C₆)alkyl and R² is hydrogen. These compounds are referred to in Scheme I as compounds of the formula “IA”.

Referring to Scheme 6, the compound of formula (28) is reacted with excess potassium carbonate and one equivalent of tosyl chloride in acetone, at a temperature from about 0° C. to about 80° C., preferably at the reflux temperature of the reaction mixture. A compound of the formula RX, wherein R is (C₁-C₆)alkyl and X is iodo, chloro or bromo, is then added to the reaction mixture and the mixture is allowed to react at a temperature ranging from about 0° C. to about 80° C., preferably at the reflux temperature of the mixture. This reaction yields a compound of the formula (29). The compound of formula (29) is then converted into the corresponding compound of formula (30) by reacting it with potassium hydroxide in ethanol, using water as the solvent. This reaction can be carried out at a temperature from about room temperature to about the reflux temperature of the reaction mixture. Preferably, the reaction mixture is heated to reflux and allowed to react at that temperature.

The compound of formula (30) is then reacted with potassium carbonate and benzyl bromide in acetone, at a temperature from about room temperature to about 80° C., to form the corresponding compound of formula (31). Preferably, the reaction is conducted at about the reflux temperature. Reaction of the resulting compound of formula (31) with butyl lithium in tetrahydrofuran (THF) at about −78° C., followed by the addition of triethyl borate and allowing the reaction mixture to warm to ambient temperature, yields the corresponding phenylboronic acid derivative of formula (32).

Reacting the phenylboronic acid derivative of formula (32) with 2-bromo-6-(2,5-dimethyl-pyrrol-1-yl)-pyridine (33), sodium carbonate and tetrakis(triphenylphosphine)palladium(0) in ethanol/water or THF/water, at a temperature from about room temperature to about the reflux temperature of the reaction mixture, preferably at about the reflux temperature, yields the corresponding compound of formula (34). Alternatively, the reactant of formula (33) can be replaced with another compound of the formula

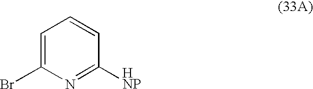

wherein P is a nitrogen protecting group such as trityl, acetyl, benzyl, trimethylacetyl, t-butoxycarbonyl, benzyloxycarbonyl, trichloroethyloxycarbonyl or another appropriate nitrogen protecting group and wherein the hydrogen that is bonded to the protected nitrogen is absent when P is a protecting group that forms a ring with the protected nitrogen, as in the case of P=2,5-dimethylpyrrolyl. Such protecting groups are well known to those of skill in the art. The above compounds of the formula (33A) are either commercially available, known in the scientific literature or easily obtaining using well known methods and reagents.

The benzyl substituent can be removed from the compound of formula (34) by reacting such compound with ammonium formate in water or a lower alcohol solvent, or in a mixture of one or more of these solvents, at a temperature from about room temperature to about the reflux temperature of the reaction mixture. This reaction is preferably carried out at the reflux temperature in the presence of about 20% palladium hydroxide on carbon. The resulting compound of formula (35) is then converted into the desired compound of formula IVA by reacting it with hydroxylamine in a solvent selected from water, lower alcohols and mixtures of these solvents, at a temperature from about room temperature to about the reflux temperature of the solvent, preferably at about the reflux temperature.

The procedure of Scheme 6 can also be used to make compounds of the formula IV wherein R¹ and R² are other than as specified above and depicted in the scheme. This can be accomplished by using a compound of the formula

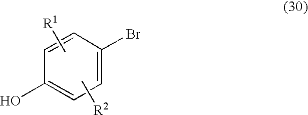

as the starting material and then carrying out the series of reactions, as described above, that are represented in Scheme 6 as reactions (30)→(31)→(32)→(33)→(34)→(35)→(IVA).

Scheme 7 illustrates a method for preparing compounds of the formula IV wherein G is hydrogen into the corresponding compounds of formula IV wherein G is other than hydrogen.

Referring to Scheme 7, a compound of the formula IVA can be converted into the corresponding compound of formula IVC by reacting it with the compound of the formula GX, wherein X is iodo, chloro, or bromo, and G is CH₂CH₂NR³R⁴, and potassium carbonate in either dimethylformamide (DMF) or acetone at a temperature from about room temperature to about the reflux temperature of the mixture, preferably at about the reflux temperature. Compounds of the formula IVC can also be formed, as illustrated in Scheme 7, as by first preparing the corresponding compounds of formula IVB and then converting them, if so desired, into the corresponding compounds of formula IVC. Compounds of formula IVB can be formed by reacting the corresponding compounds of formula IVA with a compound of the formula GX, wherein X is defined as above and G is CH₂C(═O)NR³R⁴, and potassium carbonate, in either DMF or acetone, at a temperature from about room temperature to about the reflux temperature of the reaction mixture. This reaction also is preferably carried out at about the reflux temperature.

The resulting compounds of formula of IVB can be converted into the corresponding compounds of formula IVC by reacting them with lithium aluminum hydride and aluminum chloride in a THF solvent, or with borane in THF. Other aluminum hydride reducing agents can also be used, such as diisobutyl aluminum hydride. Diborane can also be used. This reaction is generally carroid out at temperatures ranging from room temperature to about the reflux temperature of the reaction mixture, and is preferably carried out at the reflux temperature. Other appropriate sovlents include other organic ethers such as ethyl ether, dioxane and glyme, THF is preferred solvent.

Scheme 8 illustrates how certain compounds of the formula IV having different substituents R¹ and R² than are depicted in the processes of Scheme 6 can be prepared. Such compounds are prepared by a process similar to that depicted in Scheme 6, with the exception that the processes of Scheme 6 involved in the synthesis of compound (32) are replaced with those depicted in Scheme 8. Specifically, referring to Scheme 8, when R² is hydrogen and R¹ is fluoro at the ortho position, the compound of formula (36) is converted to the corresponding phenylboronic acid in a manner analogous to the conversion of compounds of the formula (31) into those of the formula (32) in Scheme (6). The resulting phenylboronic acid derivative is referred to in Scheme 8 as compound (32A). Similarly, as shown in Scheme 8, compounds of the formula IV wherein R¹ and R² are both methyl and are both at an ortho position relative to the pyridine ring, may be prepared by converting the compound of formula (37), as shown in Scheme 8, into the corresponding phenylboronic acid derivative designated as compound (32B), in a matter analogous to the conversion of compounds of formula (31) into those of the formula (32) in Scheme 6. The compounds of formulas (32A) and (32B) can then be transformed into the desired corresponding compounds of the formula IV using procedures analogous to those shown in Scheme 6.

Scheme 9 exemplifies methods of preparing compounds of the formula IV wherein G is NR³R⁴ and NR³R⁴ forms an N-methylpyrrolin-2-yl ring. Compounds of the formula IV wherein G is NR³R⁴ and NR³R⁴ forms other nitrogen containing rings can be prepared in an analogous fashion. Referring to Scheme 9, the compound of formula IVD is allowed to react with 3-methanesulfonyloxy-pyrrolidine-1-carboxylic acid tert-butyl ester to form the compound of formula (38). Other nitrogen protecting groups such as —C(═O)OCH₂C₆H₅ and COOR (wherein R is benzyl, phenyl, t-butyl or a similar group) can be used to protect the pyrrolidine nitrogen. Also, the mesylate leaving group can be replaced with another appropriate leaving group. Preferably, a catalytic amount of tetrabutylammonium iodide (TBAI) is added to the reaction mixture. This alkylation reaction is typically carried out in the presence of an alkali metal alkoxide, preferable potassium tert-butoxide, in a high boiling polar organic solvent such as dimethylsulfoxide (DMSO) or DMF, preferably DMSO. The reaction temperature can range from about 50° C. to about 100° C., and is preferably about 100° C.

Reduction of the compound of formula XII yields the compound of formula IVF. This reduction is preferably accomplished using lithium aluminum hydride as the reducing agent and tetrahydrofuran (THF) or another organic ether (eg., ethyl ether or glyme) as the solvent. Other aluminum hydride reducing agents can also be used, such as diisobutyl aluminum hydride. Diborane can also be used. The foregoing reaction is generally conducted at a temperature from about room temperature to about the reflux temperature of the reaction mixture, preferably at about the reflux temperature.

As illustrated in Scheme 10, alkylation of the compound of formula IVD with 1-(2-chloroethyl)-pyrrolidine yields the compound of formula IVE. This reaction is generally conducted in the present of a base such as cesium carbonate, potassium carbonate, or sodium carbonate, preferably cesium carbonate, in a solvent such as acetone, DMSO or acetonitrile, preferably acetone, at a temperature from about room temperature to about the reflux temperature, preferably at about the reflux temperature.

Compounds of the formula IV wherein NR³R⁴ do not form a ring can also be prepared by the method illustrated in Scheme 10 and described above for the formation of the compound of formula IVE. Structural formula IVG, depicted in Scheme 5, includes such compounds.

Scheme 11 illustrates a method of preparing the benzeneboronic acid intermediates use in the syntheses described in Schemes 6 and 8 above wherein the benzene ring of the benzeneboronic acid contains a cycloalkyl substituent. Such intermediates can be used in the processes of Schemes 6 and 8 to form compounds of the formula IV wherein one or both of R¹ and R² are cycloalkyl groups. Referring to Scheme 11, the compound of formula (39) is allowed to reflux, in the presence of magnesium metal, in THF or ethyl ether for about 8 hours, after which cyclobutanone is added to the reaction mixture. This reaction yields the compound of formula (40). Reduction of the compound of formula (40) using, for example, hydrogen gas and 10% palladium on carbon, in a lower alcohol solvent such as ethanol, at a temperature of about room temperature, yields the corresponding compound of formula (41).

Reaction of the compound of formula (41) with benzylbromide in the presence of a base such as potassium, cesium or sodium carbonate, in a solvent such as acetone, dichlorothane, chloroform or methylene chloride, at a temperature from about room temperature to about the reflux temperature of the reaction mixture, preferably at about the reflux temperature, yields the corresponding compound of formula (42).

The compound of formula (42) that was formed in the above step is then brominated by reaction with N-bromosuccinamide (NBS) and silica gel in a chlorinated hydrocarbon solvent such as carbon tetrachloride, methylene chloride or chloroform. This reaction is typically carried out at room temperature. The compound of formula (43) that is produced in this reaction can then be converted into the benzeneboronic acid derivative of formula (44) in the following manner. First, the compound of formula (43), in a solvent such as THF, is cooled to a temperature of about −78° C. to about −70° C., after which n-butyl lithium is added. After stirring the reaction mixture for about 1 hour, triethyl borate is added and the mixture is allowed to stir for an additional 1-3 hours. The benzeneboronic acid intermediate can then be isolated by methods well known to of those skilled in the art (e.g., quenching with ammonium chloride, adding water followed by concentrated hydrochloric acid, and then extracting with ethyl acetate).

Scheme 12 exemplifies a process for making compounds of the formula IV wherein G is alkenyl, as well as compounds of the formula IV wherein G is hydrogen and R² is an alkyl or alkenyl group. Referring to Scheme 12, the compound of formula IVA is converted into the corresponding compound having the formula IVH using an alkylation reaction analogous to that used to convert the compound of formula IVD into that of formula IVG in Scheme 11. Heating the resulting compound of formula IVH to about 230° C. yields the corresponding compounds of formulas IVJ and IVK. Hydrogenation of the compounds of formulas IVJ and IVK, using methods well know to those of skilled in the art (e.g., using hydrogen gas in ethanol of about 50 pounds per square inch, in the presence of 10% palladium on carbon at about room temperature) yields the corresponding alkyl derivatives of, respectively, formulas IVL and IVM. Alkylation of the compounds of formulas IVL and IVM (wherein G is hydrogen), using any of the alkylation methods described in Schemes 7, 9, and 10, and the appropriate alkylating agent, yields the corresponding desired compounds wherein G is other than hydrogen.

Scheme 13 illustrates an alternate method of preparing compounds of the formula IV wherein G is NR³R⁴(C₀-C₄) alkyl. Referring to Scheme 13, a compound of the formula (45) is reacted with bromine in acetic acid at a temperature from about 0° C. to about 60° C., preferably at about room temperature. This reaction produces the corresponding compound having a bromine substituent para to the fluoro substituent, which can then be converted into the corresponding boronic acid derivative of formula (46) as described above for the synthesis of compounds of the formula (32) (in Scheme 6) and (44) (in Scheme 11).

Addition of the 2,5-dimethylpyrroyl protecting group as described above for the synthesis of compounds of the formula (34) (in Scheme 6) yields the corresponding compound of formula (47). The compound of formula (47) is then reacted with a compound of the formula R³R⁴NOH and an alkali metal hydride, preferably sodium hydride, in a polar, organic solvent such as DMF or DMSO, preferably DMF, at a temperature between about 50° C. and about 110C, preferably at about 100° C., to form a compound that is identical to the corresponding desired compound of formula IVN, but for the presence of the 2,5-dimethylpyrrolyl protecting group. Removal of the protecting group, as described above for the preparation of compounds of the formula IVA (in Scheme 6) yields the desired compound of formula IVN.

Scheme 14 illustrates a method of synthesizing compounds of the formula I wherein G is an optionally substituted pyrrolidin-2-yl or pyrrolidin-3-yl group. Referring to Scheme 14, a compound of the formula IVA is reacted with a compound of the formula

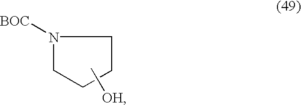

triphenylphosphine and diethylazodicarboxylate or another water soluble azodicarboxylate in THF under standard Mistsunobo reaction conditions. Typically, the reactants are combined at about 0° C. and then allowed to warm to room temperature. (If an alkyl substituent on the pyrrolidine nitrogen other than methyl is desired in the final product of formula IVP, this can be accomplished by replacing the BOC group of formula (49) with a group of the formula —C(═O)R, wherein R is the desired alkyl group).

The compound of formula (48) that is formed in the above reaction (or the corresponding —C(═O)R protected compound) can be converted into the desired product having formula IVP (or a similar compound wherein the methyl substitutuent depicted in structure IVP is replaced with another alkyl group) by reducing it. This reduction can be accomplished by reacting the product from the preceding reaction with lithium aluminum hydride and aluminum chloride in THF or borane in THF as described above for the formation of compounds of the formula IVC.

The corresponding compound of formula IV wherein the alkyl substituent on the pyrrolidine nitrogen formula IVP is replaced with hydrogen can be obtained by reacting the compound of formula (48), or an alkyl analogue of (48), as referred to above, with trifluoroacetic acid or hydrochloric acid in a solvent such as dioxane, or ether, preferably dioxane, at a temperature from about 0° C. to about reflux temperature of the reaction mixture, preferably at about the reflux temperature.

The starting materials used in the procedures of Schemes 6-14 are, the syntheses of which are not described above, either commercially available, known in the art or readily obtainable from known compounds using method that will be apparent to those skilled in the art.

The preparation of other compounds of the formula IV not specifically described in the foregoing experimental section can be accomplished using combinations of the reactions described above that will be apparent to those skilled in the art.

In each of the reactions discussed or illustrated above, pressure is not critical unless otherwise indicated. Pressures from about 0.5 atmospheres to about 5 atmospheres are generally acceptable, and ambient pressure, i.e., about 1 atmosphere, is preferred as a matter of convenience.

Compounds of the formula V and their pharmacetically acceptable salts can be prepared as described in PCT patent application PCT/IB97/01446, entitled “6-Phenylpyridyl-2-amine Derivatives”, which designates the United States and was filed on Nov. 17, 1997. The foregoing applications is incorporated herein by reference in its entirety. The foregoing application is incorporated herein by reference in its entirety.

Schemes 15-19 below illustrate methods of preparing compounds of the formula V.

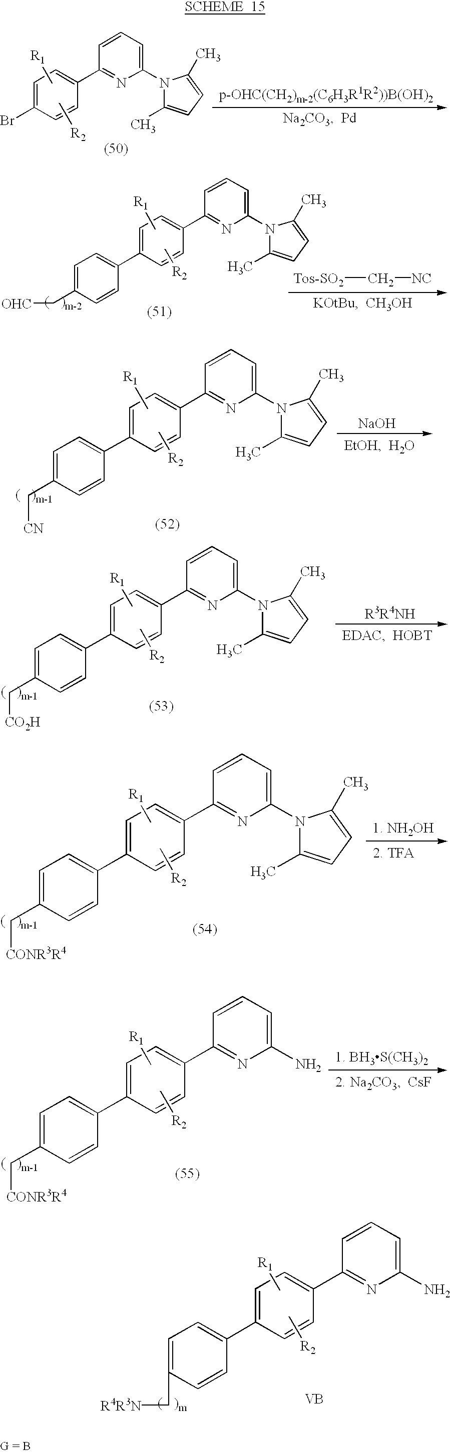

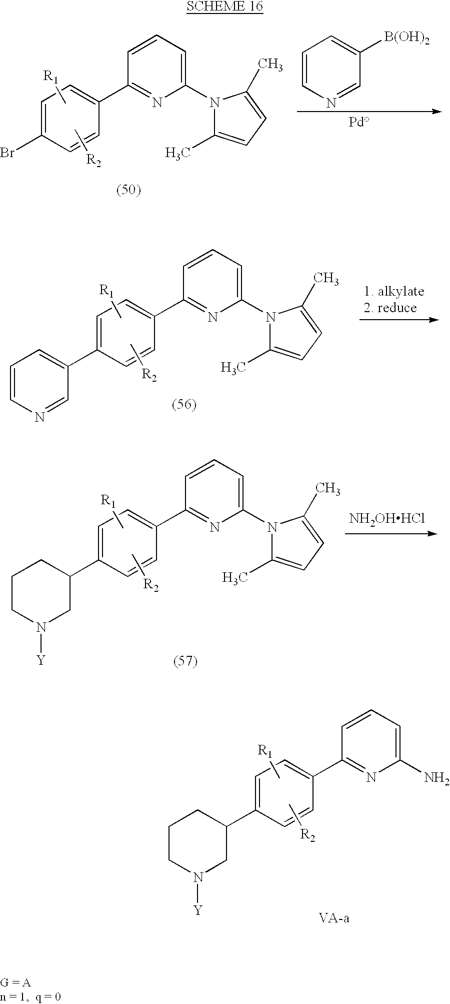

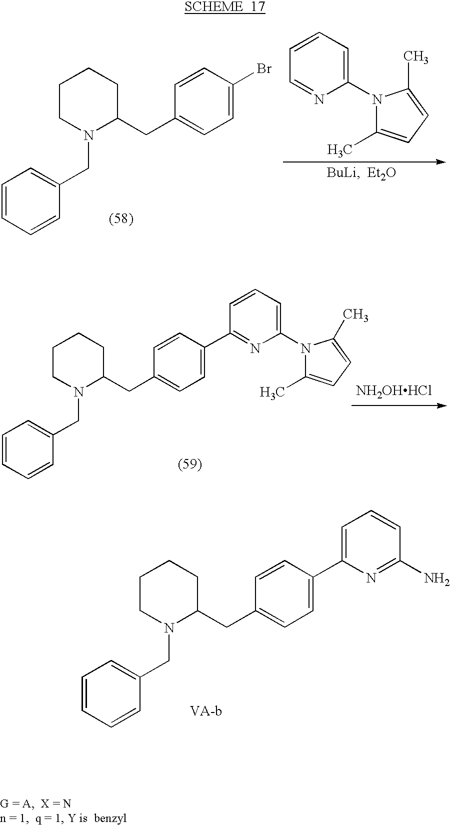

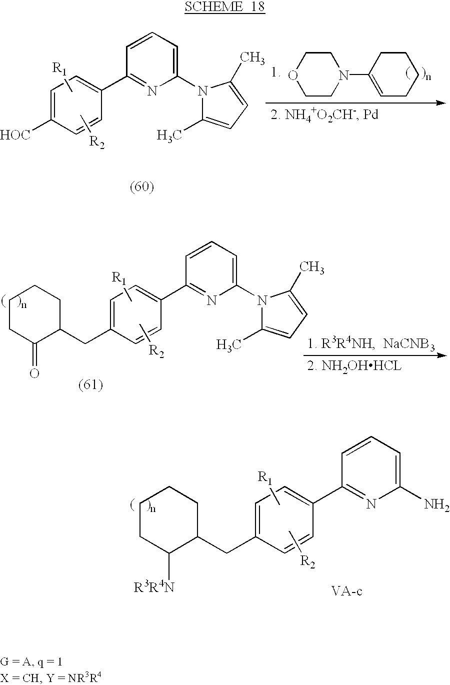

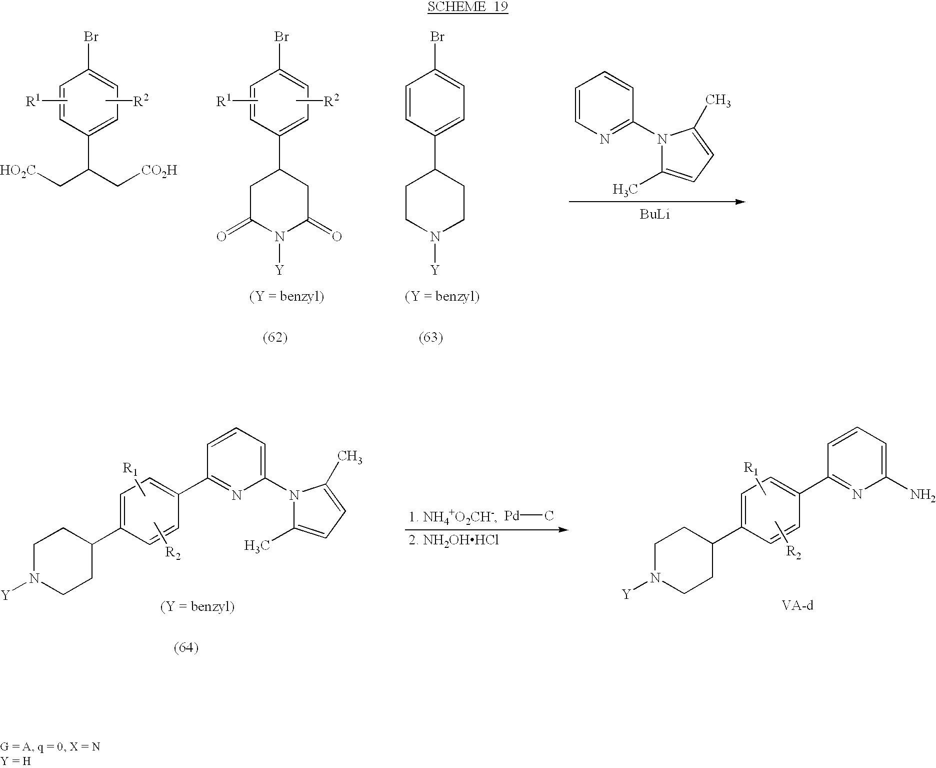

The starting materials used in the procedures of Schemes 15-19 are either commercially available, known in the art or readily obtainable from known compounds using methods that will be apparent to those skilled in the art.

Referring to Scheme 15, compound (50) is prepared by reaction of 1,4-dibromobenzene with an organolithium reagent, preferably butyl lithium, at a temperature from −100° C. to about 0° C., followed by addition to 2-(2,5-dimethylpyrrolyl)-pyridine at a temperature from about about 0° C. to about 50° C. in an ethereal solvent, preferably diethyl ether, for about 1 to 24 hours. Compound (51) is prepared by reacting (50) with a boronic acid derivative of the formula p-OHC(CH₂)ₘ₋₂(C₆H₃R¹R²)B(OH)₂ in a solvent consisting of an alcohol, preferably ethanol, optionally mixed with water and a halogenated hydrocarbon, at a temperature from about 25° C. to about 150° C., for about 1 to 24 hours, using a palladium-based catalyst, either palladium-zero or palladium-two oxidation state, typically with phosphine ligands, preferably tetrakis-triphenylphosphine palladium.

Compound (52) is prepared by reacting (51) with tosylmethylisocyanide in the presence of potassium t-butoxide and ethanol, in an ethereal solvent such as 1,2-dimethoxyethane, at a temperature from about −100° C. to about 100° C., for about 1 to 24 hours. Compound (53) is prepared from (52) by basic hydrolysis of the nitrile using an alkali metal hydroxide in an aqueous alcohol-based solvent, such as aqueous ethanol, at a temperature from about 25° C. to about 125° C., for about 30 minutes to 48 hours. Compound (54) is prepared from (53) by dehydrative coupling with ammonia, a primary or secondary amine of the formula R³R⁴NH effected by a dehydrating agent such as a carbodiimide, for example, N-ethyl-N-(dimethylaminopropyl)-carbodiimide, in a solvent that is a halogenated hydrocarbon or a N,N-dialkylamide, such as dimethylformamide, at a temperature from about 0° C. to about 100° C., for about 1 to 48 hours. Compound (55) is prepared from (54) by deblocking using hydroxylamine hydrochloride in an aqueous or alcoholic solvent, preferably aqueous ethanol, at a temperature from about 25° C. to about 100° C., for about 1 to 48 hours, and may include deblocking a protecting group such a the t-butoxycarbonyl group by reaction with trifluoroacetic acid or a related polyhalogenated acetic acid or a gaseous hydrogen halide such as HCl, in a halogenated hydrocarbon, ethereal solvent or ethyl acetate, at a temperature from about −70° C. to about 100° C., for about 10 minutes to 24 hours.

The final compound in Scheme 15, VB, wherein G=B, is prepared by reduction of (55) with borane, a trialkyl borane, alane, or lithium aluminum hydride in an ethereal solvent, such as ethyl ether or tetrahydrofuran, at a temperature from about −100° C. to about 100° C., for about 30 minutes to 24 hours, and optionally using cesium fluoride and an alkali metal or alkaline earth carbonate in an aqueous alcoholic solvent, at a temperature from about 25° C. to about 125° C. for 1 to 72 hours.

Referring to Scheme 16, compound (56) is prepared from (50) by reaction with 3-pyridyl boronic acid and a palladium catalyst, in either the palladium-zero or palladium-two oxidation state, with ligands typically comprised of trialkyl or triaryl phosphines, such as tetrakis-triphenylphosphine palladium, in an aqueous alcoholic solvent at a temperature from about 25° C. to about 125° C. for about 1 to 48 hours. Compound (57) is prepared from (56) by alkylation with an alkyl or aralkyl halide or sulfonate, in an ethereal, alcoholic , aqueous alcoholic, or dialkylamine-based solvent, such as dimethylformamide, at a temperature from about 0° C. to about 125° C. for about 30 minutes to 72 hours, followed by reduction with a borohydride- or aluminum hydride-based reagent, such as sodium borohydride, in an ethereal, alcoholic, or aqueous-alcoholic solvent, typically methanol, at a temperature from about 0° C. to about 125° C. for about 1 to 72 hours. The final compound in Scheme 16, compound VA-a, where G=A, n=1, and q=0, is prepared from (57) by deblocking with hydroxylamine hydrochloride in an alcoholic or aqueous-alcoholic solvent, typically aqueous ethanol, at a temperature from about 25° C. to about 125° C. for about 1 to 72 hours.

In the process of Scheme 16, the preferred value of Y in formulas (57) and VA-a is benzyl. Compounds of the formula VA-a wherein Y is benzyl can be converted into the corresponding compounds wherein Y is other than benzyl by debenzylation using hydrogen or ammonium formate in the presence of a noble metal catalyst, such as palladium, in an ethereal, halogenated hydrocarbon, alcoholic, or aqueous alcoholic solvent, at a temperature from 0° C. to 100° C. for a time from 30 minutes to 24 hours, followed by reductive amination with with an alkyl or aralkyl aldehyde in the presence of a borohydride-based reagent such as sodium cyanoborohydride or sodium triacetoxyborohydride, in an ethereal, halogenated hydrocarbon, alcoholic, or aqueous-alcoholic solvent, at a temperature from 0° C. to 100° C. for a time from 1 to 72 hours.

Referring to Scheme 17, compound (58) is prepared by reductive amination of 2-(4-bromophenylmethyl)-piperidine with benzaldehyde and a borohydride-based reagent such as sodium cyanoborohydride or sodium triacetoxyborohydride, in an ethereal, halogenated hydrocarbon, alcoholic, or aqueous-alcoholic solvent, at a temperature from about 0° C. to about 100° C. for about 1 to 72 hours. Compound (59) is prepared from compound (58) by reaction of compound (58) with an organolithium reagent, typically butyl lithium, followed by addition of the resulting organolithium reagent to 2-(2,5-dimethylpyrrolyl)-pyridine, in an ethereal solvent such as ethyl ether, at a temperature from about −70° C. to about 100° C. for about 30 minutes to 48 hours. The final compound in Scheme 17, IA-b, wherein G=A, n=1, q=1 and Y is benzyl, is prepared from compound (59) by deblocking with hydroxylamine hydrochloride in an alcoholic or aqueous-alcoholic solvent, typically aqueous ethanol, at a temperature from about 25° C. to about 125° C. for about 1 to 72 hours.

Compounds of the formula IA-b can be converted into the corresponding compounds wherein Y is other than benzyl using the procedure described above for converting compounds of the formula IA-a into the analogous compounds wherein Y is other than benzyl.

Referring to Scheme 18, compound (60) is prepared from 6-bromo-2-(2,5-dimethylpyrrolyl)-pyridine and 4-formylphenylboronic acid in the presence of a palladium catalyst, in either the palladium-zero or palladium-two oxidation state, with ligands typically comprised of trialkyl or triaryl phosphines, such as tetrakis-triphenylphosphine palladium, in an aqueous alcoholic solvent, at a temperature from about 25° C. to about 125° C. for about 1 to 48 hours. Compound (61) is then prepared from (60) by reaction of (60) with the enamine of a ketone or aldehyde, typically the morpholine or pyrrolidine enamine, in a aromatic hydrocarbon, hydrocarbon, or halogenated hydrocarbon solvent, preferably toluene, at a temperature from about 25° C. to about 150° C. for about 1 to 72 hours, followed by an aqueous hydrolysis step, typically with aqueous hydrochloric acid, and then reduction with hydrogen or ammonium formate in the presence of a noble metal catalyst, such as palladium, in an ethereal, halogenated hydrocarbon, alcoholic, or aqueous alcoholic solvent, at a temperature from about 0° C. to about 100° C. for about 30 minutes to 24 hours. The final compound in Scheme 18, VA, where G=A, q=1, X=CH, and Y=NR³R⁴, is prepared by reductive amination of compound (61) with ammonia, a primary amine, or a secondary amine in the presence of a borohydride-based reagent such as sodium cyanoborohydride or sodium triacetoxyborohydride, in an ethereal, halogenated hydrocarbon, alcoholic, or aqueous-alcoholic solvent, at a temperature from about 0° C. to about 100° C. for about 1 to 72 hours, followed by deblocking with hydroxylamine hydrochloride in an alcoholic or aqueous-alcoholic solvent, typically aqueous ethanol, at a temperature from about 25° C. to about 125° C. for about 1 to 72 hours.

Referring to Scheme 19, compound (62) is prepared from 3-(4-bromophenyl)-glutaric acid by dehydration with acetic anhydride or a similar dehydrating reagent, followed by reaction with benzylamine in a hydrocarbon, aromatic hydrocarbon, or halogenated hydrocarbon solvent, at a temperature from about 25° C. to about 180° C. for about 1 to 48 hours, followed by dehydration with acetic anhydride, or a similar dehydrating reagent, at a temperature from about 25° C. to about reflux for about 1 to 48 hours. Compound (63) is prepared by reduction of (64) with borane, borane methyl sulfide, alane, or lithium aluminum hydride in an ethereal or hydrocarbon solvent, at a temperature from about 0° C. to about 100° C. for about 30 minutes to 48 hours. Compound (64) is prepared from compound (63) by reaction of compound (63) with an organolithium reagent, typically butyl lithium, followed by addition of the resulting organolithium reagent to 2-(2,5-dimethylpyrrolyl)-pyridine, in an ethereal solvent, such as ethyl ether, at a temperature from about −70° C. to about 100° C. for about 30 minutes to 48 hours. The final compound in Scheme 19, VA-d, where G=A, Y=H, q=0, and X=N, is prepared by debenzylation of compound (64) using hydrogen or ammonium formate in the presence of a noble metal catalyst, such as palladium, in an ethereal, halogenated hydrocarbon, alcoholic, or aqueous alcoholic solvent, at a temperature from 0° C. to 100° C. for a time from 30 minutes to 24 hours, followed by deblocking with hydroxylamine hydrochloride in an alcoholic or aqueous-alcoholic solvent, typically aqueous ethanol, at a temperature from about 25° C. to about 125° C. for about 1 to 72 hours.

Compounds of the formula VA-d, which are prepared using the procedures of Scheme 19, can be converted into the analogous compounds wherein Y is alkyl or aralkyl, by reductive amination with an alkyl or aralkyl aldehyde in the presence of a borohydride-based reagent such as sodium cyanoborohydride or sodium triacetoxyborohydride, in an ethereal, halogenated hydrocarbon, alcoholic, or aqueous-alcoholic solvent, at a temperature from 0° C. to 100° C. for a time from 1 to 72 hours.

The preparation of other compounds of the formula V not specifically described in the foregoing experimental section can be accomplished using combinations of the reactions described above that will be apparent to those skilled in the art.

In each of the reactions discussed or illustrated above, pressure is not critical unless otherwise indicated. Pressures from about 0.5 atmospheres to about 5 atmospheres are generally acceptable, and ambient pressure, i.e., about 1 atmosphere, is preferred as a matter of convenience.

Compounds of the formula VI can be prepared as described below and in the U.S. provisional application of John A. Lowe, III that was filed on Jun. 3, 1998 and is entitled “2-Aminopyridines Containing Fused Ring Substituents”. The foregoing application is incorporated herein by reference in its entirety.

Scheme 20 below illustrates a method of preparing compounds of the formula VI.

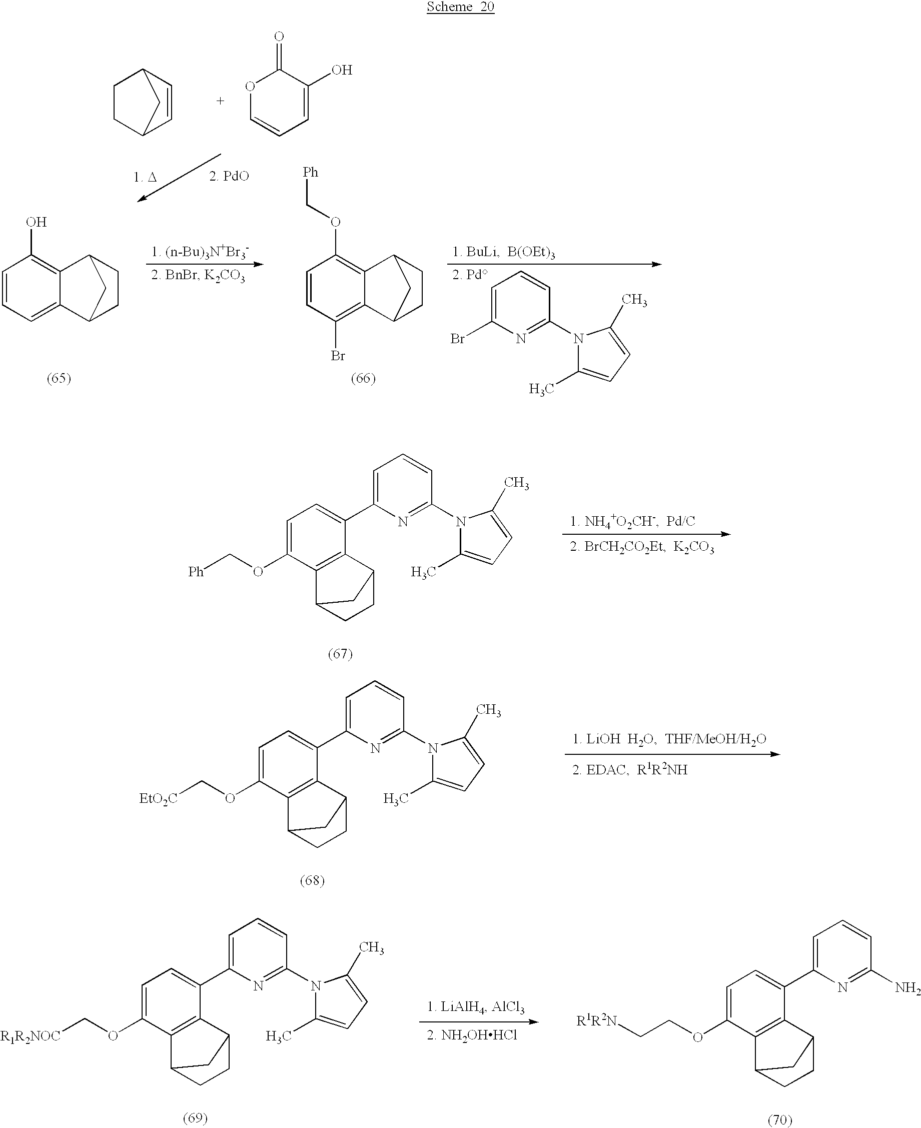

Referring to Scheme 20, the compound of formula (65) is prepared by reaction of norbornylene and 2-hydroxypyrone followed by aromatization with palladium oxide, according to the procedure described in Syn. Commun., 5, 461, (1975). It is then reacted with tetrabutylammonium tribromide in 1,2-dichloroethane at about room temperature for about 10 minutes to about 10 hours. The product of this reaction is then treated with benzyl bromide and potassium carbonate in a solvent such as acetonitrile, at about the reflux temperature of the reaction mixture for about 1 to 48 hours, to form the compound of formula (66).

The compound of formula (66) is then converted into 5-benzyloxy-1,2,3,4-tetrahydro-1,4-methano-naphthalene-8-boronic acid by cooling the compound of formula III to about −70° C. in dry tetrahydrofuran (THF), and adding a solution of n-butyl lithium to it. The resulting solution is then treated with triethyl borate and allowed to warm to room temperature for about 1 to 48 hours to form 5-benzyloxy-1,2,3,4-tetrahydro-1,4-methano-naphthalene-8-boronic acid. Reaction of 5-benzyloxy-1,2,3,4-tetrahydro-1,4-methano-naphthalene-8-boronic acid with 6-bromo-2-(2,5-dimethylpyrrolyl)pyridine in an ethanol solvent, in the presence of sodium carbonate and tetrakistriphenylphosphine palladium, at about the reflux temperature for about 1 to 48 hours of the reaction mixture, yields the compound of formula (67).

The compound of formula (67) can be converted into the compound of formula V using the following two step process. The compound of formula (67) is reacted with ammonium formate and ten percent palladium on carbon, in an ethanol solvent, at about the reflux temperature of the reaction mixture, for about 10 minutes to about 10 hours to yield the analogous compound to that having formula (67), wherein the benzyloxy group of formula (67) is replaced with a hydroxy group. The compound of formula (68) is then formed by reacting the above hydroxy derivative with 2-bromoethylacetate and potassium carbonate in acetonitrile at about the reflux temperature of the reaction mixture for about 1 to 48 hours.

Basic hydrolysis of the compound of formula (68), followed by reaction with N-ethyl-N-3-dimethylaminopropylcarbodiimide (EDAC) and the appropriate compound having the formula R¹R²NH yields the desired compound of the formula (69). The base hydrolysis is typically carried out using an alkali metal or alkaline earth metal hydroxide in a mixture of THF, methanol and water at about room temperature for about 1 to 48 hours. The reaction with the appropriate compound of the formula R¹R²NH and N-ethyl-N-dimethylaminopropyl carbodiimide (EDAC) is conducted in the presence of a base. Examples of suitable bases are those selected from trialkylamines, alkali metal carbonates and alkaline earth metal carbonates. This reaction is typically conducted in a solvent such as acetonitrile, methylene chloride or N,N-dimethylformamide (DMF), at a temperature from about room temperature to about 100° C., preferably at about room temperature for about 1 to 48 hours. Preferably, the reaction is conducted in the presence of a catalytic additive such as N-hydroxysuccinamide or hydroxybenzotriazole.

The compound of formula (69) can be converted into the desired compound of formula I as follows. The compound of formula (69) is reduced to form the corresponding compound wherein the carbonyl group is replaced by a methylene group, after which the 2,5-dimethylpyrrolyl protecting group is removed. The reduction can be carried out using methods well known to those of skill in the art, for example, using lithium aluminum hydride in tetrahydrofuran, with or without aluminum chloride, or using borane methyl sulfide in tetrahydrofuran, at a temperature of about −78° C. to about reflux, preferably at about −70° C. to room temperature for about 1 to about 24 hours.

Removal of the 2,5-dimethylpyrrolyl protecting group can be accomplished by reaction with hydroxylamine hydrochloride. This reaction is generally carried out in an alcoholic or aqueous alcoholic solvent (preferably, using ethanol as the alcohol), at a temperature from about room temperature to about the reflux temperature of the reaction mixture, preferably at about the reflux temperature, for about 8 to about 72 hours.

Compounds of the formula VI wherein there is a heteroatom in one of the bridging rings can be prepared in an analogous fashion, starting with the appropriate compound that is analogous to that of formula (65), wherein the unsubstituted bridged ring of formula (65) is replaced by a bridged ring comprising a heteroatom.

The preparation of other compounds of the formula VI not specifically described in the foregoing experimental section can be accomplished using combinations of the reactions described above that will be apparent to those skilled in the art.

In each of the reactions discussed or illustrated above, pressure is not critical unless otherwise indicated. Pressures from about 0.5 atmospheres to about 5 atmospheres are generally acceptable, and ambient pressure, i.e., about 1 atmosphere, is preferred as a matter of convenience.

The compounds of formulas I-VI that are basic in nature are capable of forming a wide variety of different salts with various inorganic and organic acids. Although such salts must be pharmaceutically acceptable for administration to animals, it is often desirable in practice to initially isolate a compound of the formula I, II, III, IV, V or VI from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the active base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained.

The compounds of formulas I, II, III, IV, V and VI, and their pharmaceutically acceptable salts, are useful as NOS inhibitors i.e., they possess the ability to inhibit the NOS enzyme in mammals, and therefore they are able to function as therapeutic agents in the treatment of the aforementioned disorders and diseases in an afflicted mammal.

The ability of compounds of formulas I-VI to inhibit NOS may be determined using procedures described in the literature. The ability of compounds of the formulae I to inhibit endothelial NOS may be determined by using the procedures described by Schmidt et al. in Proc. Natl. Acad. Sci. U.S.A., 88, pp. 365-369 (1991) and by Pollock et al., in Proc. Natl. Acad. Sci. U.S.A., 88, pp.10480-10484 (1991). The ability of compounds of the formulae I to inhibit inducible NOS may be determined using the procedures described by Schmidt et al., in Proc. Natl. Acad, Sci. U.S.A., 88 pp. 365-369 (1991) and by Garvey et al. in J. Biol. Chem., 269, pp. 26669-26676 (1994). The ability of the compounds of the formulae I to inhibit neuronal NOS may be determined using the procedure described by Bredt and Snyder in Proc. Natl. Acad. Sci. U.S.A., 87, 682-685 (1990).

The compounds of formula I-VI and their pharmaceutically acceptable salts can be administered via either the oral, parenteral or topical routes. In general, these compounds are most desirably administered, when used as the single active agent for the treatment of psoriasis, sleep disorders or cognitive deficits or disorders, in dosages ranging from about 0.01 to about 250 mg per day, in single or divided doses (i.e., from 1 to 4 doses per day), although variations will necessarily occur depending upon the species, weight and condition of the subject being treated and the particular route of administration chosen. However, a dosage level that is in the range of about 0.07 mg to about 21 mg per kg of body weight per day is most desirably employed. Variations may nevertheless occur depending upon the species of animal being treated and its individual response to said medicament, as well as on the type of pharmaceutical formulation chosen and the time period and interval at which such administration is carried out. In some instances, dosage levels below the lower limit of the aforesaid range may be more than adequate, while in other cases still larger doses may be employed without causing any harmful side effect, provided that such larger doses are first divided into several small doses for administration throughout the day.

The compounds of formulas I-VI may be administered alone or in combination with pharmaceutically acceptable carriers or diluents by either of the three routes previously indicated, and such administration may be carried out in single or multiple doses. More particularly, such therapeutic agents can be administered in a wide variety of different dosage forms, i.e., they may be combined with various pharmaceutically acceptable inert carriers in the form of tablets, capsules, lozenges, troches, hard candies, powders, sprays, creams, salves, suppositories, jellies, gels, pastes, lotions, ointments, aqueous suspensions, injectable solutions, elixirs, syrups, and the like. Such carriers include solid diluents or fillers, sterile aqueous media and various non-toxic organic solvents, etc. Moreover, oral pharmaceutical compositions can be suitably sweetened and/or flavored. In general, the therapeutically-effective compounds of this invention are present in such dosage forms at concentration levels ranging from about 5.0% to about 70% by weight.

For oral administration, tablets containing various excipients such as microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tableting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration, solutions of a compound of the formula I, II, III, IV, V or VI, or a pharmaceutically acceptable salt thereof, in either sesame or peanut oil or in aqueous propylene glycol may be employed. The aqueous solutions should be suitably buffered (preferably pH greater than 8) if necessary and the liquid diluent first rendered isotonic. These aqueous solutions are suitable for intravenous injection purposes. The oily solutions are suitable for intraarticular, intramuscular and subcutaneous injection purposes. The preparation of all these solutions under sterile conditions is readily accomplished by standard pharmaceutical techniques well known to those skilled in the art.

Additionally, it is also possible to administer the compounds of formulas I-VI topically when treating inflammatory conditions of the skin, and this may be done by way of creams, jellies, gels, pastes, patches, ointments and the like, in accordance with standard pharmaceutical practice.

This invention relates both to methods of treating an inflammatory disorder in which the antiinflammatory compound and the NOS inhibiting compound are administered together, as part of the same pharmaceutical composition, and to methods in which these two active agents are administered separately as part of an appropriate dose regimen designed to obtain the benefits of the combination therapy. The appropriate dose regimen, the amount of each dose administered, and specific intervals between doses of each active agent will depend on the subject being treated, and the source and severity of the condition. Generally, in carrying out the methods of this invention, the NOS inhibiting compound will be administered to an average 70 kg adult human in an amount ranging from about 0.01 to about 10 mg per kg body weight of the subject being treated per day, in single or divided doses, preferably from about 1 to about 3 mg/kg, and the antiinflammatory agent will be administered in an amount ranging from about 0.2 to about 30 mg per kg body weight of the subject being treated per day, in single or divided doses. Variations may nevertheless occur depending upon the species of animal being treated and its individual response to said medicament, as well as on the type of pharmaceutical formulation chosen and the time period and interval at which such administration is carried out. In some instances, dosage levels below the lower limit of the aforesaid range may be more than adequate, while in other cases still larger doses may be employed without causing any harmful side effect, provided that such larger doses are first divided into several small doses for administration throughout the day.

This invention relates both to methods of treating chronic or acute pain in which the analgesic compound and the NOS inhibiting compound are administered together, as part of the same pharmaceutical composition, and to methods in which these two active agents are administered separately as part of an appropriate dose regimen designed to obtain the benefits of the combination therapy. The appropriate dose regimen, the amount of each dose administered, and specific intervals between doses of each active agent will depend on the subject being treated, and on the source and severity of the condition. Generally, in carrying out the methods of this invention, the NOS inhibiting compound will be administered to an average 70 kg adult human in an amount ranging from about 0.01 to about 10 mg per kg body weight of the subject being treated per day, in single or divided doses, preferably from about 1 to about 3 mg/kg, and the analgesic agent will be administered in an amount ranging from about 0.01 to about 1 mg per kg body weight of the subject being treated per day, in single or divided doses, preferably from about 1 to about 10 mg per day. Variations may nevertheless occur depending upon the species of animal being treated and its individual response to said medicament, as well as on the type of pharmaceutical formulation chosen and the time period and interval at which such administration is carried out. In some instances, dosage levels below the lower limit of the aforesaid range may be more than adequate, while in other cases still larger doses may be employed without causing any harmful side effect, provided that such larger doses are first divided into several small doses for administration throughout the day.

This invention relates both to methods of treating migraine, cluster and other headaches in which the 5HT_{1D} agonist and the NOS inhibiting compound are administered together, as part of the same pharmaceutical composition, and to methods in which these two active agents are administered separately as part of an appropriate dose regimen designed to obtain the benefits of the combination therapy. The appropriate dose regimen, the amount of each dose administered, and specific intervals between doses of each active agent will depend on the subject being treated, and the source and severity of the condition. Generally, in carrying out the methods of this invention, the NOS inhibiting compound will be administered to an average 70 kg adult human in an amount ranging from about 0.01 to about 10 mg per kg body weight of the subject being treated per day, in single or divided doses, preferably from about 1 to about 3 mg/kg, and the 5HT_{1D} agonist will be administered in an amount ranging from about 1 to about 100 mg per day, in single or divided doses, preferably from about 5 to about 50 mg per day. Variations may nevertheless occur depending upon the species of animal being treated and its individual response to said medicament, as well as on the type of pharmaceutical formulation chosen and the time period and interval at which such administration is carried out. In some instances, dosage levels below the lower limit of the aforesaid range may be more than adequate, while in other cases still larger doses may be employed without causing any harmful side effect, provided that such larger doses are first divided into several small doses for administration throughout the day.

The present invention is illustrated by the following examples. It will be understood, however, that the invention is not limited to the specific details of these examples. Melting points are uncorrected. Proton nuclear magnetic resonance spectra (¹H NMR) and ¹³C nuclear magnetic resonance spectra were measured for solutions in deuterochloroform (CDCl₃) or in CD₃OD or CD₃SOCD₃ and peak positions are expressed in parts per million (ppm) downfield from tetramethylsilane (TMS). The peak shapes are denoted as follows: s, singlet; d, doublet; t, triplet; q, quartet, m, multiplet, b, broad.

### EXAMPLE 1

### 1-[4-(6-AMINO-PYRIDIN-2-YL)-3-ISOPROPOXY-PHENYL]-2-(4-PHENETHYL-PIPERAZIN-1-YL)-ETHANOL

A. N-t-Butylcarbonyl-6-(2-isopropoxy-4-formylphenyl)-pyridin-2-ylamine

To a 100 mL round-bottomed flask equipped with condenser and N₂ inlet were added 4.85 g (11.97 mmol) N-t-butylcarbonyl-6-(2-isopropoxy-4-bromomethylphenyl)-pyridin-2-ylamine (from Example 1E above), 3.35 g (23.95 mmol) hexamethylene tetramine, and 30 mL chloroform, and the reaction refluxed for 2 hours. The reaction was concentrated and taken up in 24 mL of 1:1 acetic acid:water and refluxed for 5 hours. The reaction was cooled, adjusted to pH 10 with aqueous sodium hydroxide solution, and extracted into ethyl acetate. The organic phase was washed with brine, dried over sodium sulfate, and evaporated. The residue was chromatographed on silica gel using hexane/ethyl acetate as eluant to afford 2.995 g (74%) of a white solid.

¹H-NMR (δ, CDCl₃): 1.32 (m, 15H), 4.68 (septet, J=6, 1H), 7.47 (s, 1H), 7.51 (d, J=8, 1H), 7.64 (m, 1H), 7.72 (t, J=8, 1H), 7.90 (d, J=8, 1H), 8.05 (bs, 1H), 8.20 (d, J=8, 1H), 9.99 (s, 1H).

MS (%): 341 (parent+1, 100).

B. N-t-Butylcarbonyl-6-(2-isopropoxy-4-oxiranylphenyl)-pyridin-2-ylamine

To a 100 mL round-bottomed flask equipped with condenser and N₂ inlet were added 2.99 g (8.79 mmol) N-t-butylcarbonyl-6-(2-isopropoxy-4-formylphenyl)-pyridin-2-ylamine, 1.79 g (8.79 mmol) trimethylsulfonium iodide, 0.98 g (17.59 mmol) powdered potassium hydroxide, 44 mL acetonitrile, and 0.5 mL water. The reaction was heated to 60° C. for 2.5 hours, then cooled, filtered, and evaporated. The yellow oil was used directly, 3.3 g (˜100%).

¹H-NMR (δ, CDCl₃): 1.27 (d, J=6, 6H), 1.32 (s, 9H), 2.76 (m, 1H), 3.15 (m, 1H), 3.87 (m, 1H), 4.54 (septet, 1H), 6.87 (s, 1H), 6.97 (d, J=8, 1H), 7.58 (m, 1H), 7.69 (m, 2H), 8.05 (bs, 1H), 8.13 (d, J=8, 1H).

MS (%): 355 (parent+1, 100).

C. 1-[N-t-Butylcarbonyl-4-(6-amino-pyridin-2-yl)-3-isopropoxy-phenyl]-2-(4-phenethyl-piperazin-1-yl)-ethanol

To a 25 mL round-bottomed flask equipped with condenser and N₂ inlet were added 300 mg (0.847 mmol) N-t-butylcarbonyl-6-(2-isopropoxy-4-oxiranylphenyl)-pyridin-2-ylamine, 193 mg (1.017 mmol) N-phenethylpiperazine, 9 mL acetonitrile, and 0.85 mL water. The reaction was heated to 80° C. for 20 hours, cooled, and partitioned between ethyl acetate and aqueous sodium bicarbonate. The organic phase was separated, washed with brine, dried over sodium sulfate, and evaporated. The residue was chromatographed on silica gel using methanol/methylene chloride/ammonium hydroxide as eluant to afford 283 mg (62%) of an off-white foam.

¹H-NMR (δ, CDCl₃): 1.27 (d, J=6, 6H), 1.31 (s, 9H), 2.4-2.9 (m, 15H), 4.56 (septet, J=6, 1H), 4.75 (m, 1H), 6.99 (d, J=8, 1H), 7.06 (s, 1H), 7.1-7.3 (m, 5H), 7.58 (d, J=8, 1H), 7.67 (m, 2H), 8.08 (bs, 1H), 8.13 (d, J=8, 1H). ¹³C-NMR (δ, CDCl₃): 22.05, 27.45, 33.53, 39.71, 53.18, 60.36, 65.95, 68.41, 70.99, 111.54, 112.10, 118.26, 121.18, 126.01, 128.34, 128.61, 130.80, 137.67, 140.09, 144.34, 150.98, 154.29, 155.47, 176.99.

MS (%): 545 (parent+1, 100).

D. 1-[4-(6-Amino-pyridin-2-yl)-3-isopropoxy-phenyl]-2-(4-phenethyl-piperazin-1-yl)-ethanol

To a 25 mL round-bottomed flask equipped with condenser and N₂ inlet were added 283 mg (0.52 mmol) 1-[N-t-butycarbonyl-4-(6-amino-pyridin-2-yl)-3-isopropoxy-phenyl]-2-(4-phenethyl-piperazin-1-yl)-ethanol, 5 mL dioxane, and 10 mL 10% aqueous sodium hydroxide solution. The reaction was refluxed 3 days, cooled, poured into water, and extracted into ethyl acetate. The organic phase was washed with brine, dried over sodium sulfate, and evaporated. The residue was chromatographed on silica gel using methanol/methylene chloride/ammonium hydroxide as eluant to afford 203 mg (86%) of an oil, which was converted to the hydrochloride salt using HCl in tetrahydrofuran, mp 148-165° C.

¹H-NMR (δ, CDCl₃): 1.27 (d, J=6, 6H), 2.6-2.9 (m, 15H), 4.48 (bs, 2H), 4.52 (septet, J=6, 1H), 4.74 (m, 1H), 6.385 (d, J=8, 1H), 6.97 (d, J=8, 1H), 7.03 (s, 1H), 7.1-7.3 (m, 6H), 7.41 (t, J=8, 1H), 7.70 (d, J=8, 1H).

¹³C-NMR (δ, CDCl₃): 22.16, 33.62, 53.03, 53.27, 60.45, 66.04, 68.57, 71.19, 106.47, 112.56, 115.62, 118.46, 126.09, 128.42, 128.70, 129.75, 130.97, 137.27, 140.22, 143.81, 154.35, 155.52, 158.01.

MS (%): 461 (parent+1, 100).

Anal. Calc'd. for C₂₈H₃₆N₄O₂.3HCl.2H₂O: C, 55.49; H, 7.15; N, 9.24. Found: C, 55.50; H, 7.38; N, 8.97.

### EXAMPLE 2

### 6-[2-ISOPROPOXY-(N-(2-METHYL)PROPYL)-4-(PYRROLIDIN-3-YL)-PHENYL]-PYRIDIN-2-YLAMINE

A. N-t-Butylcarbonyl-6-(2-fluoro-4-bromomethylphenyl)-pyridin-2-ylamine

To a 250 mL round-bottomed flask equipped with condenser and N₂ inlet were added 5.0 g (17.48 mmol) N-t-butylcarbonyl-6-(2-fluoro-4-methylphenyl)-pyridin-2-ylamine (Example 2B), 4.36 g (24.47 mmol) N-bromosuccinimide, 10 mg azobisdi-(1,1-dimethylcyclohexyl)nitrile, and 85 mL carbon tetrachloride. The reaction was refluxed under a heat lamp for 30 min, cooled, and filtered. The filtrate was concentrated and chromatographed on silica gel using hexane/ethyl acetate as eluant to afford 5.36 g (52%) of the product as an oil, which was crystallized from isopropanol to give mp 97-100° C.

¹H-NMR (δ, CDCl₃): 1.32 (s, 9H), 4.46 (s, 2H), 7.18 (d, J=11.5, 1H), 7.24 (d, J=8, 1H), 7.49 (d, J=8, 1H), 7.74 (t, J=8, 1H), 7.88 (t, J=8, 1H), 8.06 (bs, 1H), 8.21 (d, J=8, 1H).

¹³C-NMR (δ, CDCl₃): 27.52, 31.90, 39.85, 112.92, 116.82, 117.07, 120.37, 120.47, 124.99, 125.03, 126.75, 131.17, 131.20, 138.87, 140.42, 140.51, 150.80, 151.47, 158.99, 161.48,177.15.

MS (%): 366 (parent+1, 100).

Anal. Calc'd. for C₁₇H₁₈N₂OFBr: C, 55.90; H, 4.97; N, 7.46. Found: C, 55.57; H, 4.79; N, 7.46.

B. N-t-Butylcarbonyl-6-(2-fluoro-4-formylphenyl)-pyridin-2-ylamine

To a 125 mL round-bottomed flask equipped with condenser and N₂ inlet were added 5.35 g (14.66 mmol) N-t-butylcarbonyl-6-(2-fluoro-4-bromomethylphenyl)-pyridin-2-ylamine, 36 mL chloroform, and 4.10 g (29.32 mmol) hexamethylenetetramine. The reaction was refluxed 5 hours, cooled, and evaporated. The residue was taken up in 29 mL 50% aqueous acetic acid, and refluxed 16 hours. The reaction was cooled, taken up in ethyl acetate, and washed with aqueous sodium hydroxide solution and brine, dried over sodium sulfate and evaporated. The residue was chromatographed on silica gel using hexane/ethyl acetate as eluant to afford 3.49 g (67%) of an oil.

¹H-NMR (δ, CDCl₃): 1.325 (s, 9H), 7.56 (m, 1H), 7.62 (d, J=11, 1H), 7.7-7.8 (m, 2H), 8.10 (m, 2H), 8.26 (d, J=8, 1H), 9.99 (s, 1H).

¹³C-NMR (δ, CDCl₃): 27.41, 39.78, 113.65, 116.41, 116.66, 120.67, 120.77, 125.66, 131.63, 137.84, 138.93, 149.83, 151.60,159.35,161.86, 177.14, 190.54.

MS (%): 301 (parent+1, 100).

Anal. Calc'd. for C₁₇H₁₇N₂O₂F: C, 67.99; H, 5.71; N, 9.33. Found: C, 67.62; H, 5.67; N, 9.50.

C. Diethyl-2-fluoro-4-[N-t-butylcarbonyl-6-pyridin-2-ylamine]benzylidenemalonate

To a 125 mL round-bottomed flask equipped with N₂ inlet were added 2.65 g (8.83 mmol) N-t-butylcarbonyl-6-(2-fluoro-4-formylphenyl)-pyridin-2-ylamine, 1.41 g (8.83 mmol) diethyl malonate, 45 mL benzene, 40 mg (0.44 mmol) piperidine, and 10 mg benzoic acid. The reaction was refluxed 3 days, cooled, and poured into water and ethyl acetate. The organic layer was washed with 1 N hydrochloric acid, aqueous sodium bicarbonate solution, and brine, dried over sodium sulfate, and evaporated. The residue was chromatographed on silica gel using hexane/ethyl acetate as eluant to afford the product as a yellow oil, 3.14 g (80%), which was crystallized from 2-propanol, mp 97-100° C.

¹H-NMR (δ, CDCl₃): 1.32 (m, 15H), 4.29 (q, J=7, 2H), 4.34 (q, J=7, 2H), 7.24 (d, J=12, 1H), 7.32 (d, J=8, 1H), 7.53 (d, J=7, 1H), 7.67 (s, 1H), 7.75 (t, J=8, 1H), 7.96 (t, J=8, 1H), 8.05 (bs, 1H), 8.22 (d, J=8, 1H).

¹³C-NMR (δ, CDCl₃): 13.94, 14.12, 27.51, 39.85, 61.89, 61.97, 113.27, 116.75, 117.00, 120.53, 120.63, 125.63, 125.66, 127.77, 131.10, 131.13, 135.09, 135.17, 138.95, 139.89, 150.29, 151.53,159.04, 161.55,163.76,166.20, 177.16.

MS (%): 443 (parent+1, 100).

Anal. Calc'd. for C₂₄H₂₇N₂O₅F: C, 65.15; H, 6.15; N, 6.33. Found: C, 64.88; H, 6.18; N, 6.59.

D. Ethyl-3-[2-fluoro-4-(N-t-butylcarbonyl-6-pyrid in-2-ylamine)]phenyl-3-cyano-propionate

To a 125 mL round-bottomed flask equipped with condenser and N₂ inlet were added 3.12 mg (7.05 mmol) diethyl-2-fluoro-4-[N-t-butylcarbonyl-6-pyridin-2-ylamine]benzylidenemalonate and 100 mL ethanol. To the stirring solution was added a solution of 460 mg (7.05 mmol) potassium cyanide in 1.8 mL water, and the reaction stirred at room temperature for 3 days, then heated for 38 hours at 60° C. The reaction was cooled and quenched with dilute hydrochloric acid, then taken up in ethyl acetate and washed with acid and brine, dried over sodium sulfate, and evaporated. The residue was chromatographed on silica gel using hexane/ethyl acetate as eluant to afford 1.88 g (67%) of an oil.

¹H-NMR (δ, CDCl₃): 1.24 (t, J=7, 3H), 1.32 (s, 9H), 2.93 (AB_{q}, J=8, Δv=58, 2H), 4.17 (m, 2H), 4.33 (t, J=7, 1H), 7.19 (d, J=11, 1H), 7.26 (d, J=8, 1H), 7.48 (m, 1H), 7.75 (t, J=8, 1H), 7.94 (t, J=8, 1H), 8.05 (bs, 1H), 8.225 (d, J=8, 1H).

¹³C-NMR (δ, CDCl₃): 14.0, 27.4, 32.5, 39.6, 39.8, 61.6, 113.0, 115.4, 115.7, 119.2, 120.6, 123.4, 127.6, 127.7, 131.7, 137.0, 138.9, 150.3, 151.4, 159.1, 161.6, 168.7, 177.1.

MS (%): 398 (parent+1, 100).

E. N-t-Butylcarbonyl-6-[2-fluoro-4-(2-oxo-pyrrolidin-3-yl )-phenyl]-pyridin-2-ylamine

To a 125 mL Paar bottle were added 1.88 g (4.73 mmol) ethyl-3-[2-fluoro-4-(N-t-butylcarbonyl-6-pyridin-2-ylamine)]phenyl-3-cyano-propionate, 35 mL ethanol, 1 g 10% palladium-on-carbon and 2 mL 6 N hydrochloric acid. The reaction was shaken under 40 p.s.i. hydrogen for 20 hours, filtered through Celite, and the filtrate evaporated. The residue was taken up in ethyl acetate, washed with aqueous sodium hydroxide, dried over sodium sulfate, and evaporated. The residue was taken up in 35 mL dry toluene, treated with 3.5 mL triethylamine, and heated at reflux for 18 hours. The reaction was then cooled, washed with dilute aqueous hydrochloric acid and brine, dried over sodium sulfate, and evaporated. The residue was chromatographed on silica gel using hexane/ethyl acetate as eluant to afford 394 mg (23%) of a solid, mp 162-165° C.

¹H-NMR (δ, CDCl₃): 1.31 (s, 9H), 2.59 (AB_{q}, J=8, Δv=112, 2H), 3.27 (m, 1H), 3.68 (m, 2H), 7.01 (d, J=12, 1H), 7.10 (d, J=8, 1H), 7.19 (s, 1H), 7.44 (m, 1H), 7.73 (t, J=8, 1H), 7.84 (t, J=8, 1H), 8.20 (d, J=8, 1H), 8.23 (bs, 1H).

¹³C-NMR (δ, CDCl₃): 27.465, 37.8, 39.6, 39.9, 49.2, 112.9, 114.6, 114.8, 120.2, 120.3, 122.7, 125.6, 128.2, 129.0, 131.3, 138.9, 145.7, 150.9, 151.6, 15.2, 161.7, 177.3, 177.5.

MS (%): 356 (parent+1, 100).

Anal. Calc'd. for C₂₀H₂₂N₃O₂F: C, 67.59; H, 6.24; N, 11.82. Found: C, 67.49; H, 6.37; N, 11.76.

F. 6-[2-Fluoro-4-(2-oxo-pyrrolidin-3-yl)-phenyl]-pyridin-2-ylamine

The above material was deblocked using 6 N hydrochloric acid at 90° C. for 18 hours, followed by treatment with N-ethyl,N-isopropylcarbodiimide and N-hydroxybenztriazole with triethylamine and 4-dimethylaminopyridine in acetonitrile at room temperature for 2 days. The reaction was worked up with ethyl acetate and water, dried over sodium sulfate, and evaporated. The residue was chromatographed on silica gel using methanol/methylene chloride as eluant to afford a solid, mp 185-188° C., 167 mg (47%).

¹H-NMR (δ, CDCl₃): 2.49 (AB_{q}, J=8, Δv=108, 2H), 3.22 (m, 1H), 3.60 (m, 2H), 4.90 (bs, 2H), 6.38 (d, J=8, 1H), 6.87 (m, 2H), 6.97 (d, J=8, 1H), 7.35 (t, J=8, 1H), 7.59 (t, J=8, 1H).

¹³C-NMR (δ, CDCl₃): 37.6, 39.3, 49.1, 108.0, 114.1, 114.4, 122.4, 126.3, 131.0, 138.2, 144.6, 150.6, 158.6, 158.8, 161.3, 177.9.

MS (%): 272 (parent+1, 100).

G. 6-[2-Fluoro-4-(pyrrolidin-3-yl)-phenyl]-pyridin-2-ylamine

To a 25 mL round-bottomed flask equipped with N₂ inlet were added 160 mg (0.59 mmol) 6-[2-fluoro-4-(2-oxo-pyrrolidin-3-yl)-phenyl]-pyridin-2-ylamine and 8 mL dry tetrahydrofuran. The solution was cooled to −70° C., and 5.9 mL (5.9 mmol) of a 1.0 M solution was lithium aluminum hydride in tetrahydrofuran was added. The reaction was warmed to room temperature and stirred 2 days. The reaction was carefully quenched with dilute aqueous sodium hydroxide solution, then taken up in ethyl acetate and aqueous sodium hydroxide solution, and the combined organic layer washed with water, dried over sodium sulfate, and evaporated to afford a crude oil, which was used directly in the next step.

¹H-NMR (δ, CDCl₃): 1.8-2.0 and 2.2-2.4 (m, 2H), 2.6-3.7 (m, 5H), 4.80 (bs, 2H), 6.41 (d, J=8, 1H), 6.92 (m, 2H), 7.01 (d, J=8, 1H), 7.21 (d, J=8, 1H), 7.395 (t, J=8, 1H), 7.66 (t, J=8, 1H), 7.71 (m, 1H).

MS (%): 258 (100, parent+1)

H. 6-[2-Fluoro-(N-(2-methyl)propyl)-4-(pyrrolidin-3-yl)-phenyl]-pyridin-2-ylamine

To a 25 mL round-bottomed flask equipped with N₂ inlet were added 151 mg (0.587 mmol) 6-[2-fluoro-4-(pyrrolidin-3-yl)-phenyl]-pyridin-2-ylamine, 85 mg (1.175 mmol) isobutyraldehyde, 74 mg (1.175 mmol) sodium cyanoborohydride, and 6 mL methanol. The reaction was stirred at room temperature for 2 hours, poured into dilute hydrochloric acid, and washed with ethyl acetate. The aqueous layer was adjusted to pH 12 with 1 N aqueous sodium hydroxide solution and extracted with ethyl acetate. The organic layer was dried over sodium sulfate and evaporated, and the residue chromatographed on silica gel using methanol/methylene chloride to afford 25 mg (%) of an oil.

¹H-NMR (δ, CDCl₃): 0.94 (d, J=6, 6H), 1.7-1.9 (m, 2H), 2.32 (m, 3H), 2.55 (m, 1H), 2.74 (m, 2H), 2.98 (m, 1H), 3.37 (m, 1H), 4.49 (bs, 2H), 6.44 (d, J=8, 1H), 7.05 (d, J=12, 1H), 7.11 (m, 2H), 7.46 (t, J=8, 1H), 7.79 (t, J=8, 1H).

¹³C-NMR (δ, CDCl₃): 21.0, 27.2, 33.0, 42.7, 54.7, 61.9, 64.7, 107.2, 114.6, 114.7, 123.2, 125.4, 130.5, 137.9, 148.4, 151.6, 158.1, 159.0, 161.5.

MS (%): 314 (parent+1, 100).

I. 6-[2-Isopropoxy-(N-(2-methyl)propyl)-4-(pyrrolid in-3-yl)-phenyl]-pyridin-2-ylamine

To a 25 mL round-bottomed flask equipped with condenser and N₂ inlet were added 24 mg (0.077 mmol) 6-[2-fluoro-(N-(2-methyl)propyl)-4-(pyrrolidin-3-yl)-phenyl]-pyridin-2-ylamine and 3 mL dry dimethylformamide. The solution was heated to 80° C., and 46 mg (0.767 mmol) 2-propanol. 37 mg (0.920 mmol) sodium hydride (60% dispersion in oil), The reaction was stirred at 100° C. for 18 hours, then cooled and evaporated. The residue was treated with dioxane and 1 N aqueous sodium hydroxide solution to cleave some N-formylated byproduct at room temperature for 18 hours. The reaction was partitioned between 0.5 N aqueous sodium hydroxide solution and ethyl acetate, and the organic layer washed with brine, dried over sodium sulfate, and evaporated. The residue was chromatographed by preparative plate silica gel chromatography using methanol/methylene chloride/ammonia as eluant to afford 24 mg (89%) of an oil, which was converted to the hydrochloride salt, mp 118-138° C.

¹H-NMR (δ, CDCl₃): 0.96 (d, J=7, 6H), 1.25 (d, J=6, 6H), 1.8 (m, 1H), 1.9 (m, 1H), 2.4 (m, 3H), 2.64 (m, 1H), 2.85 (m, 2H), 3.07 (m, 1H), 3.38 (m, 1H), 4.45 (m, 3H), 6.395 (d, J=8, 1H), 6.92 (m, 2H), 7.22 (t, J=8, 1H), 7.42 (t, J=7, 1H), 7.64 (d, J=8, 1H).

¹³C-NMR (δ, CDCl₃): 21.0, 22.2, 27.2, 33.1, 43.2, 55.0, 62.0, 64.75, 71.2, 106.4, 114.5, 115.6, 119.9, 128.7, 131.0, 137.3, 146.4, 154.4, 155.4, 157.9.

MS (%): 354 (parent+1, 100).

1. A method of treating an inflammatory disorder in a mammal, comprising adminstering to said mammal:
(a) a NOS inhibiting compound of the formula I, II, III, IV, V or VI, as defined in the specification, or a pharmaceutically acceptable salt thereof; and
(b) a compound that exhibits antiiflammatory activity, or a pharmaceutically acceptable salt thereof;
wherein the active agents “a” and “b” above are present in amounts that render the combination of the two agents effective in treating such disorder.

2. A method of treating chronic or acute pain in a mammal, comprising adminstering to said mammal:
(a) a NOS inhibiting compound of the formula I, II, III, IV, V or VI, as defined in the specification, or a pharmaceutically acceptable salt thereof; and
(b) a narcotic analgesic compound or a pharmaceutically acceptable salt thereof;
wherein the active agents “a” and “b” above are present in amounts that render the combination of the two agents effective in treating chronic or acute pain.

3. A pharmaceutical composition for treating an inflammatory disorder in a mammal, comprising:
(a) a compound that exhibits antiinflammatory activity, or a pharmaceutically acceptable salt thereof;
(b) a NOS inhibiting compound of the formula I, II, III, IV, V or VI, as defined in the specification, or pharmaceutically acceptable salt thereof; and
(c) a pharmaceutically acceptable carrier;
wherein the active agents “a” and “b” are present in such composition in amounts that render the combination of the two agents effective in treating such disorder.

4. A pharmaceutical composition for treating chronic or acute pain in a mammal, including a human, comprising:
(a) a NOS inhibiting compound of the formula I, II, III, IV, V or VI, as defined in the specification, or pharmaceutically acceptable salt thereof;
(b) a narcotic analgesic compound or a pharmaceutically acceptable salt thereof; and
(c) a pharmaceutically acceptable carrier;
wherein the active agents “a” and “b” are present in such composition in amounts that render the combination of the two agents effective in treating such disorder.

5. A pharmaceutical composition for treating a condition selected from migraine, cluster and other vascular headaches in a mammal, comprising:
(a) a NOS inhibiting compound or pharmaceutically acceptable salt thereof; and
(b) a serotonin-1D (5HT_{1D}) receptor agonist or a pharmaceutically acceptable salt thereof; and
(c) a pharmaceutically acceptable carrier;
wherein the active agents “a” and “b” are present in such composition in amounts that render the combination of the two agents effective in treating such condition.

6. A method for treating a condition selected from migraine, cluster and other vascular headaches in a mammal, comprising administering to said mammal:
(a) a NOS inhibiting compound or pharmaceutically acceptable salt thereof; and
(b) a serotonin-1D (5HT_{1D}) receptor agonist or a pharmaceutically acceptable salt thereof;
wherein the active agents “a” and “b” are present in such composition in amounts that render the combination of the two agents effective in treating such condition.

7. A pharmaceutical composition for treating a condition selected from the group consisting of sleep disorders, psoriasis and cognitive deficits or disorders in a mammal, comprising an amount of a NOS inhibiting compound of the formula I, II, III, IV, V or VI, as defined in the specification, that is effective in treating such condition, and a pharmaceutically acceptable carrier.

8. A method of treating a condition selected from the group consisting of sleep disorders, psoriasis and cognitive deficits or disorders in a mammal, comprising administering to said mammal an amount of a NOS inhibiting compound of the formula I, II, III, IV, V or VI, as defined in the specification, that is effective in treating or preventing such condition.

9. A pharmaceutical composition for treating or preventing a condition selected from the group consisting of sleep disorders, psoriasis and cognitive deficits or disorders in a mammal, comprising a NOS inhibiting effective amount of a compound of the formula I, II, III, IV, V or VI, as defined in the specification, or a pharmaceutically acceptable salt thereof, and a pharmaceutically acceptable carrier.

10. A method of treating a condition selected from the group consisting of sleep disorders, psoriasis and cognitive deficits or disorders in a mammal, comprising administering to said mammal a NOS inhibiting effective amount of a compound of the formula I, II, III, IV, V or VI, as defined in the specification, or a pharmaceutically acceptable salt thereof.
